(19) United States
(12) Patent Application Publication
(10) Pub. No.: US 2011/0077534 A1
(43) Pub. Date: Mar. 31, 2011

(54) BLOOD PRESSURE INFORMATION MEASUREMENT DEVICE CAPABLE OF OBTAINING INDEX FOR DETERMINING DEGREE OF ARTERIOSCLEROSIS

(75) Inventors: Tatsuya Kobayashi, Shiga (JP); Toshihiko Ogura, Kyoto (JP); Hironori Sato, Shiga (JP); Toshihiko Abe, Tokyo (JP); Hideaki Yoshida, Kyoto (JP)

(73) Assignee: OMRON HEALTHCARE CO., LTD., Kyoto (JP)

(21) Appl. No.: 12/993,699

(22) PCT Filed: Apr. 28, 2009
(86) PCT No.: PCT/JP2009/058341

§ 371 Date: Nov. 19, 2010

(30) Foreign Application Priority Data
May 27, 2008 (JP) 2008-138385

(51) Int. Cl. A61B 5/022

(52) U.S. Cl. 600/490

(57) ABSTRACT

A blood pressure information measurement device includes an air bladder wrapped around a central side of a measurement portion for pulse wave measurement and an air bladder wrapped at a peripheral side for blood pressure measurement. The air bladder for blood pressure measurement is increased to a pressure higher than a systolic blood pressure, and a blood pressure is measured. An internal pressure of the air bladder is maintained, a pulse wave is measured in avascularized state, and a feature point needed for calculating an index for determining an arteriosclerosis degree is extracted from the pulse wave. When the feature point is not extracted from the pulse wave, the internal pressure of the air bladder for blood pressure measurement is reduced to a pressure lower than the systolic blood pressure, a pulse wave in non-avascularized state is measured, and a feature point is extracted from the pulse wave.

### TECHNICAL FIELD

This invention relates to a blood pressure information measurement device and an index acquisition method. More particularly, the invention relates to an apparatus for measuring blood pressure information by using a cuff including a fluid bag and a method for obtaining an index for determining a degree of arteriosclerosis from the blood pressure information.

### BACKGROUND ART

Measuring blood pressure information such as blood pressure and pulse wave is useful for determining a degree of arteriosclerosis.

Conventionally, for example, Japanese Unexamined Patent Publication No. 2000-316821 (hereinafter referred to as Patent Document 1) discloses an apparatus for determining the degree of arteriosclerosis by checking a velocity at which a pulse wave ejected from a heart (hereinafter referred to as PWV: pulse wave velocity). The pulse wave transmission velocity increases as the degree of arteriosclerosis advances. Therefore, the PWV serves as an index for determining the degree of arteriosclerosis. The PWV is calculated by attaching cuffs and the like for measuring pulse waves at at least two or more positions such as an upper arm and a lower limb, measuring the pulse waves at a time, and calculating the PWV based on a difference of times at which the pulse waves emerge at respective positions and a length of an artery between the two points at which the cuffs and the like for measuring the pulse waves are attached. The PWV differs according to measurement positions. Typical examples of PWVs include baPWV obtained from measuring positions of an upper arm and an ankle and cfPWV obtained from measuring positions of a carotid artery and a femoral artery.

As a technique for determining the degree of arteriosclerosis from a pulse wave at an upper arm, Japanese Unexamined Patent Publication No. 2007-44362 (hereinafter referred to as Patent Document 2) discloses a technique having a double structure including a blood pressure measuring cuff and a pulse wave measuring cuff.

Japanese Unexamined Patent Publication No. 2004-113593 (hereinafter referred to as Patent Document 3) discloses a technique for separating an ejection wave ejected from a heart and a reflection wave reflected by a stiffened portion in an artery and an iliac artery branching portion, and determining the degree of arteriosclerosis based on amplitude differences, amplitude ratios, and emerging time differences thereof.

Patent Document 1: Japanese Unexamined Patent Publication No. 2000-316821

Patent Document 2: Japanese Unexamined Patent Publication No. 2007-44362

Patent Document 3: Japanese Unexamined Patent Publication No. 2004-113593

### SUMMARY OF THE INVENTION

However, in order to measure a PWV using the apparatus disclosed in Patent Document 1, it is necessary to attach the cuffs and the like to at least two positions such as an upper arm and a lower limb as described above. Therefore, it is difficult to easily measure a PWV at home even when the apparatus disclosed in Patent Document 1 is used.

In contrast, Patent Document 2 discloses a technique for determining a degree of arteriosclerosis from a pulse wave at an upper arm. The apparatus disclosed in Patent Document 2 has the double structure including the blood pressure measuring cuff and the pulse wave measuring cuff. However, with the pulse wave measuring cuff alone, a reflection from a periphery is overlapped. Accordingly, a reflection wave may not be correctly separated. Therefore, it is difficult to determine the degree of arteriosclerosis with high accuracy.

Further, depending on a subject, it may be difficult to find a feature point for determining the degree of arteriosclerosis based on a pulse wave obtained by avascularizing a peripheral side which is measured by the apparatus disclosed in Patent Document 3.

One or more embodiments of the present invention provides a blood pressure information measurement device and an index acquisition method capable of obtaining an index for accurately determining the degree of arteriosclerosis from measured blood pressure information.

According to an aspect of the present invention, a blood pressure information measurement device includes a first fluid bag and a second fluid bag, a first sensor and a second sensor for respectively measuring internal pressures of the first fluid bag and the second fluid bag, a first adjusting unit for adjusting the internal pressure of the second fluid bag, and a control unit for controlling calculation for calculating an index for determining a degree of arteriosclerosis and adjustment of the first adjusting unit, wherein the control unit performs calculation for detecting a first pulse wave of a measurement portion based on a change of the internal pressure of the first fluid bag in a first state in which the first fluid bag is wrapped around the measurement portion, the second fluid bag is wrapped at a peripheral side with respect to the first fluid bag, and the second fluid bag presses the peripheral side with respect to the measurement portion around which the first fluid bag is wrapped with an internal pressure higher than a systolic blood pressure, calculation for detecting a second pulse wave based on a change of the internal pressure of the first fluid bag in a second state in which the first fluid bag is wrapped around the measurement portion, the second fluid bag is wrapped at a peripheral side with respect to the first fluid bag, and the second fluid bag presses the peripheral side with respect to the measurement portion around which the first fluid bag is wrapped with an internal pressure lower than at least the systolic blood pressure, and calculation for calculating the index using at least one of a first feature point extracted from the first pulse wave and a second feature point extracted from the second pulse wave.

According to another aspect of the present invention, a blood pressure information measurement device includes a first fluid bag and a second fluid bag, a first sensor and a second sensor for respectively measuring internal pressures of the first fluid bag and the second fluid bag, a first adjusting unit for adjusting the internal pressure of the second fluid bag, and a control unit for controlling calculation for calculating an index for determining a degree of arteriosclerosis and adjustment of the first adjusting unit, wherein the control unit performs calculation for detecting a pulse wave of a measurement portion based on a change of the internal pressure of the first fluid bag in which the first fluid bag is wrapped around the measurement portion, the second fluid bag is wrapped at a peripheral side with respect to the first fluid bag, and the second fluid bag presses the peripheral side with respect to the measurement portion around which the first fluid bag is wrapped, calculation for comparing a systolic blood pressure with the internal pressure of the second fluid bag when the pulse wave is detected, and determining whether the detected pulse wave is a first pulse wave detected in a first state in which the peripheral side of the measurement portion is pressed while the internal pressure of the second fluid bag is higher than the systolic blood pressure or a second pulse wave detected in a second state in which the peripheral side of the measurement portion is pressed while the internal pressure of the second fluid bag is lower than at least the systolic blood pressure, and calculation for calculating the index using at least one of a first feature point extracted from the first pulse wave and a second feature point extracted from the second pulse wave.

According to still another aspect of the present invention, an index acquisition method for obtaining an index for determining a degree of arteriosclerosis from a pulse wave measured by a blood pressure information measurement device, wherein the blood pressure information measurement device includes a first fluid bag and a second fluid bag, a first sensor and a second sensor for respectively measuring internal pressures of the first fluid bag and the second fluid bag, and a first adjusting unit for adjusting the internal pressure of the second fluid bag, and the index acquisition method includes the steps of controlling the internal pressure of the second fluid bag such that the internal pressure of the second fluid bag attains a pressure higher than a systolic blood pressure, detecting a first pulse wave of a measurement portion based on a change of the internal pressure of the first fluid bag in a first state in which the first fluid bag is wrapped around the measurement portion, the second fluid bag is wrapped at a peripheral side with respect to the first fluid bag, and the second fluid bag presses the peripheral side with respect to the measurement portion around which the first fluid bag is wrapped with an internal pressure higher than the systolic blood pressure, calculating the index from the first pulse wave, performing control to reduce the internal pressure of the second fluid bag in a case where the index is not calculated from the first pulse wave, detecting a second pulse wave of the measurement portion based on a change of the internal pressure of the first fluid bag in a state in which the first fluid bag is wrapped around the measurement portion, the second fluid bag is wrapped at a peripheral side with respect to the first fluid bag, and the second fluid bag presses the peripheral side with respect to the measurement portion with a pressure lower than at least the systolic blood pressure, and calculating the index from the second pulse wave.

By using the blood pressure information measurement device according to one or more embodiments of the present invention, it is possible to obtain an index for accurately determining the degree of arteriosclerosis based on the measured blood pressure information.

### BRIEF DESCRIPTION OF THE DRAWINGS

FIG. 1 is a perspective view illustrating a specific example of external appearance of a measurement device according to a first embodiment.

FIG. 2A is a diagram illustrating a specific example of a measuring posture when the measurement device according to the first embodiment is used to measure blood pressure information.

FIG. 2B is a schematic cross sectional view illustrating a specific example of a configuration of an arm band according to the first embodiment.

FIG. 3 is a diagram illustrating a relationship between a pulse wave waveform and an index for determining a degree of arteriosclerosis.

FIG. 4 is a diagram illustrating a specific example of correlation between a PWV and a time difference Tr between an ejection wave and a reflection wave.

FIG. 5 is a diagram representing a pulse wave measured when a peripheral side is avascularized and a pulse wave measured when the peripheral side is not avascularized.

FIG. 6 is a diagram illustrating functional blocks of the measurement device according to the first embodiment.

FIG. 7 is a flowchart illustrating a first specific example of a measuring operation performed by the measurement device according to the first embodiment.

FIG. 8 is a diagram illustrating pressure change within each air bladder during the measuring operation performed by the measurement device according to the first embodiment.

FIG. 9 is a flowchart illustrating a second specific example of the measuring operation performed by the measurement device according to the first embodiment.

FIG. 10 is a flowchart illustrating a third specific example of the measuring operation performed by the measurement device according to the first embodiment.

FIG. 11 is a flowchart illustrating a fourth specific example of the measuring operation performed by the measurement device according to the first embodiment.

FIG. 12 is a diagram illustrating functional blocks of the measurement device according to a second embodiment.

FIG. 13 is a flowchart illustrating a first specific example of a measuring operation performed by the measurement device according to the second embodiment.

FIG. 14 is a diagram illustrating pressure change within each air bladder during the measuring operation performed by the measurement device according to the second embodiment.

FIG. 15 is a flowchart illustrating a second specific example of the measuring operation performed by the measurement device according to the second embodiment.

FIG. 16 is a flowchart illustrating a modification of the second specific example of the measuring operation performed by the measurement device according to the second embodiment.

FIG. 17 is a diagram illustrating pressure change within each air bladder during the measuring operation performed by the measurement device according to the second embodiment.

FIG. 18 is a flowchart illustrating a third specific example of the measuring operation performed by the measurement device according to the second embodiment.

FIG. 19A is a diagram illustrating a specific example of a measuring posture when a measurement device according to a third embodiment is used to measure blood pressure information.

FIG. 19B is a schematic cross sectional view illustrating a specific example of a configuration of an arm band according to the third embodiment.

FIG. 20 is a diagram illustrating functional blocks of the measurement device according to the third embodiment.

FIG. 21 is a flowchart illustrating a first specific example of a measuring operation performed by the measurement device according to the third embodiment.

FIG. 22 is a diagram illustrating pressure change within each air bladder during the measuring operation performed by the measurement device according to the third embodiment.

FIG. 23 is a flowchart illustrating a second specific example of the measuring operation performed by the measurement device according to the third embodiment.

FIG. 24 is a flowchart illustrating a third specific example of the measuring operation performed by the measurement device according to the third embodiment.

FIG. 25 is a flowchart illustrating a fourth specific example of the measuring operation performed by the measurement device according to the third embodiment.

### DETAILED DESCRIPTION

Embodiments of the present invention will be hereinafter described with reference to the drawings. In embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid obscuring the invention. In the below description, the same reference numerals are attached to the same components and constituent elements. Names and functions thereof are also the same.

It should be noted that “blood pressure information” means information related to blood pressure obtained by measuring a living body. More specifically, “blood pressure information” includes a blood pressure value, pulse wave waveform, heart rate, and the like.

### First Embodiment

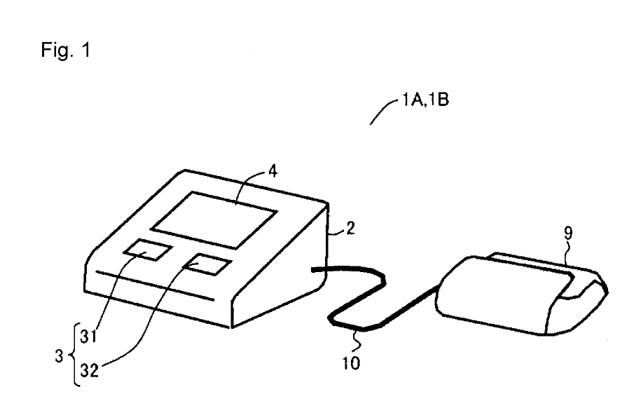

Referring to FIG. 1, a blood pressure information measurement device 1A according to a first embodiment (hereinafter abbreviated as measurement device) includes a base body 2 and an arm band 9 connected to the base body 2 and attached to an upper arm, i.e., a measurement portion. The base body 2 and the arm band 9 are connected via an air tube 10. On a front surface of the base body 2, a display unit 4 and an operation unit 3 are arranged. The display unit 4 displays various kinds of information including a measurement result. The operation unit 3 is operated to give various kinds of instructions to the measurement device 1A. The operation unit 3 includes a switch 31 operated to turn on and off a power supply and a switch 32 operated to give an instruction to start a measuring operation.

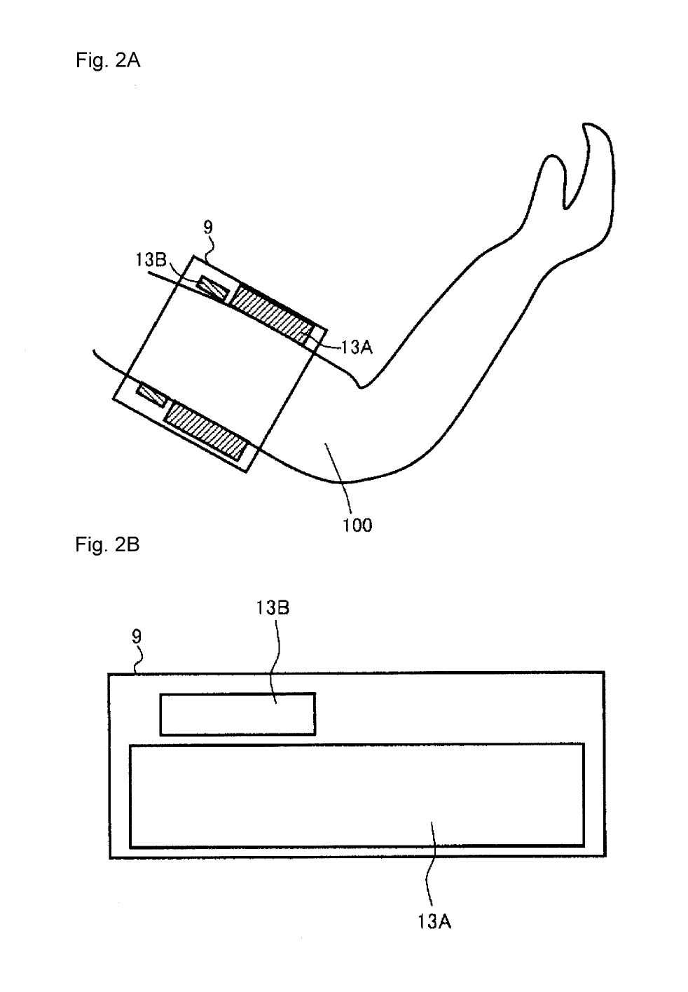

When a pulse wave is measured using the measurement device 1A, an arm band 9 is wrapped around an upper arm 100, i.e., the measurement portion, as shown in FIG. 2A. When the switch 32 is pressed down in this state, blood pressure information is measured.

Referring to FIG. 2A, the arm band 9 includes an air bladder, i.e., a fluid bag for pressing a living body. The air bladder includes an air bladder 13A, i.e., a fluid bag, used for measuring blood pressure as blood pressure information, and an air bladder 13B, i.e., a fluid bag, used for measuring a pulse wave as blood pressure information. For example, as shown in FIG. 2B, the size of the air bladder 13B is about 20 mm×200 mm. According to one or more embodiments of the present invention, an air capacity of the air bladder 13B is ⅕ or less of an air capacity of the air bladder 13A as shown in FIG. 2B.

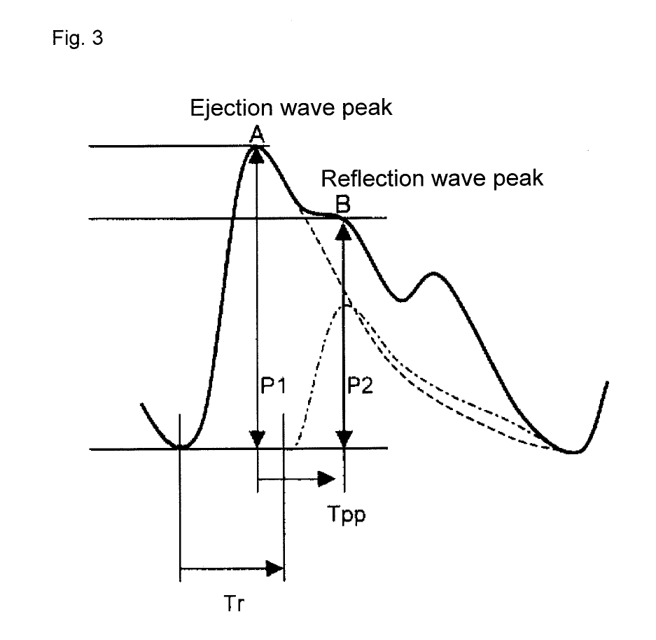

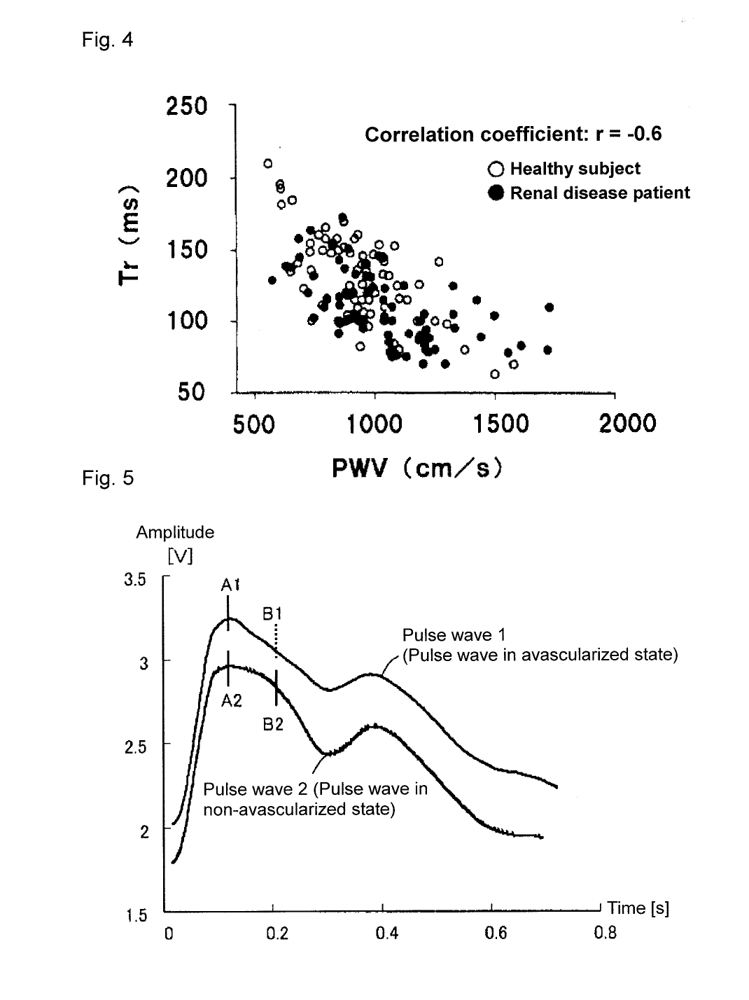

The measurement device 1A obtains an index for determining the degree of arteriosclerosis based on a pulse wave waveform, i.e., blood pressure information, obtained from one measurement portion. Examples of indexes for determining the degree of arteriosclerosis include Tpp (which is also represented as ΔTp), Tr (Traveling time to reflected wave), and AI (Augmentation Index). Tpp is an index represented by a time interval between an emerging time of a peak (maximum point) of an ejection wave, i.e., a traveling wave, and an emerging time of a peak (maximum point) of a reflection wave. In a waveform of FIG. 3, Tpp is represented by a time interval between a point A and a point B. Tr is an index represented by a time interval between an emerging time of an ejection wave and an emerging time of a reflection wave reflected by and returned from a branching point of an iliac artery when a traveling wave is reflected by the branching point. In a waveform of FIG. 3, Tr is represented by a time interval between a rising point of the ejection wave and the point A. As shown in FIG. 4, the index Tr and a PWV are related with each other. Pages 10 to 19 of “Hypertension 1992 Jul; 20 (1):” by London et al. (issued on Jul. 20, 1992) describe as follows. When a measurement portion is an upper arm, and a reflection wave is a reflection wave from an ankle, i.e., a periphery, a correlation between an index Tr and baPWV, i.e., PWV in a case where the measurement portions are the upper arm and the ankle, provides individual parameters such as height and sex. Therefore, the emerging time difference Tr can be adopted as an index for determining the degree of arteriosclerosis. This is also applicable to Tpp. AI is an index based on a feature quantity reflecting the intensity of reflection of a pulse wave mainly corresponding to arteriosclerosis. The intensity of reflection of a pulse wave is an index representing a reflection phenomenon of the pulse wave and representing the degree of ease of blood pumping and the degree of ease of receiving a blood flow volume. AI is an index represented by a ratio of a reflection wave at the maximum point with respect to an amplitude of an ejection wave, i.e., traveling wave, at the maximum point. In the waveform of FIG. 3, AI is represented as a ratio of an amplitude P2 at the point B with respect to an amplitude P1 at the point A.

In order to obtain these indexes from the measured pulse wave, it is necessary to extract a peak of the ejection wave (point A of FIG. 3) and a peak of the reflection wave (point B of FIG. 3) from the measured pulse wave. The points A and B in FIG. 3 are inflection points of the pulse wave waveform, and the points A and B will be referred to as “feature points”. The points A and B, i.e., the inflection points, are obtained by performing multi-order differentiation of the measured pulse wave waveform (for example, fourth-order differentiation).

In order to obtain the above-described feature points, i.e., the inflection points, from the pulse wave waveform obtained through measurement, it is necessary to obtain a highly accurate pulse wave waveform. Accordingly, in the first embodiment, the air bladder for pressing a living body has a double structure including two air bladders 13A, 13B arranged side by side in a direction of an artery of a measurement portion. When the arm band 9 is wrapped around the upper arm 100, the air bladder 13A is arranged at a peripheral side of the upper arm 100 (a side far from the heart). When the arm band 9 is wrapped around the upper arm 100, the air bladder 13B is arranged at a central side (a side closer to the heart). After the upper arm 100 is pressed and fixed, these air bladders 13A, 13B inflate and deflate. When the air bladder 13A inflates, the air bladder 13A is pressed onto the upper arm 100. A change of an artery pressure is detected together with an internal pressure of the air bladder 13A. Further, when the air bladder 13A inflates, the peripheral side of the artery is avascularized. When the air bladder 13B inflates in this state, an artery pressure pulse wave generated within the artery is detected in the avascularized state. That is, the pulse wave can be measured while the peripheral side is avascularized. Therefore, the pulse wave can be measured with high accuracy. As a result, feature points can be accurately obtained from the measured pulse wave waveform, and a highly accurate index can be obtained.

However, depending on a subject, it may be difficult to find feature points from a pulse wave detected by avascularizing the peripheral side. That is, when a pulse wave as shown in FIG. 5 is detected, a peak point A1 of an ejection wave is extracted from a “pulse wave 1” measured in the avascularized state. In contrast, it is difficult to find a peak point B1 of a reflection wave, and the peak point B1 is not extracted. However, a reflection wave from the peripheral side in a “pulse wave 2” measured in a non-avascularized state affects more greatly than in the avascularized state. Therefore, in the “pulse wave 2” measured in a non-avascularized state, a peak point A2 of the ejection wave as well as a peak point B2 of the reflection wave are extracted. When these pulse waves are overlaid as shown in FIG. 5, the emerging time of the point A1 and the emerging time of the point A2 are considered to be the same for the same subject. Likewise, the emerging time of the point B1 and the emerging time of the point B2 are considered be substantially the same.

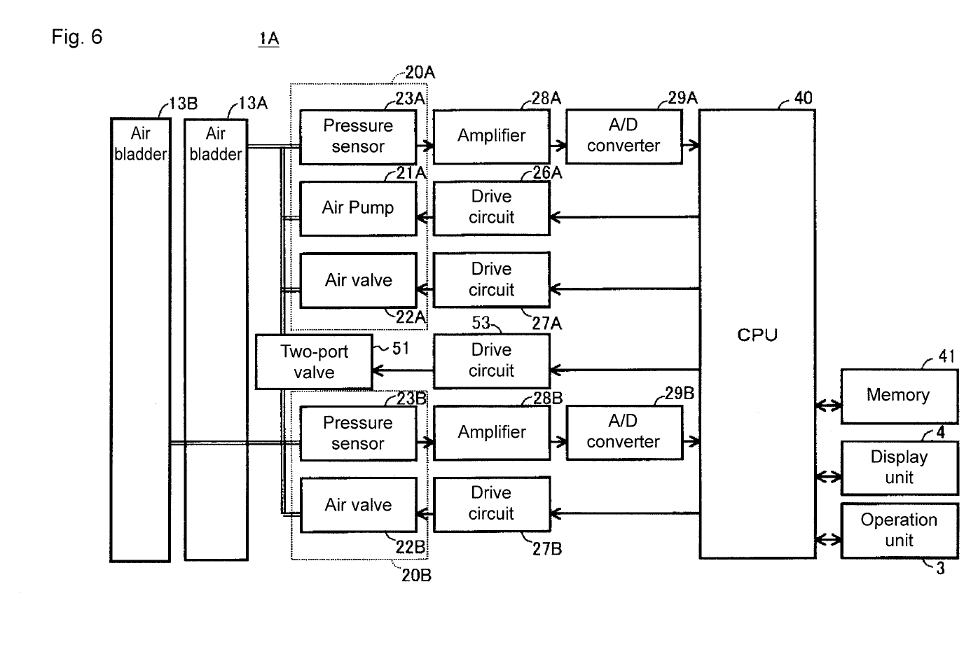

Referring to FIG. 6, the measurement device 1A includes an air system 20A connected to the air bladder 13A via the air tube 10, an air system 20B connected to the air bladder 13B via the air tube 10, and a CPU (Central Processing Unit) 40.

The air system 20A includes an air pump 21A, an air valve 22A, and a pressure sensor 23A. The air system 20B includes an air valve 22B and a pressure sensor 23B.

The air pump 21A is driven by a drive circuit 26A receiving an instruction from the CPU 40, and pumps compressed gas to the air bladder 13A. Thereby, the air bladder 13A is pressurized.

The open/close states of the air valves 22A, 22B are controlled by the drive circuits 27A, 27B receiving instructions from the CPU 40. The pressures in the air bladders 13A, 13B are controlled by controlling the open/close states of the air valves 22A, 22B.

The pressure sensors 23A, 23B respectively detect the pressures in the air bladders 13A, 13B, and output signals to amplifiers 28A, 28B according to the detected values thereof. The amplifiers 28A, 28B respectively amplifies the signals outputted from the pressure sensors 23A, 23B, and outputs the amplified signals to ND converters 29A, 29B. The A/D converters 29A, 29B respectively digitalize analog signals outputted from the amplifiers 28A, 28B, and output the digital signals to the CPU 40.

The air bladder 13A and the air bladder 13B are connected by a two-port valve 51. The two-port valve 51 is connected to a drive circuit 53, which controls opening and closing of the valve. The drive circuit 53 is connected to the CPU 40, and controls opening and closing of the above two valves of the two-port valve 51 according to a control signal given by the CPU 40.

The CPU 40 controls the air systems 20A, 20B and the drive circuit 53 based on instructions inputted to the operation unit 3 on the base body 2 of the measurement device. Measurement results are outputted to the display unit 4 and a memory 41. The memory 41 stores the measurement results. The memory 41 also stores programs executed by the CPU 40.

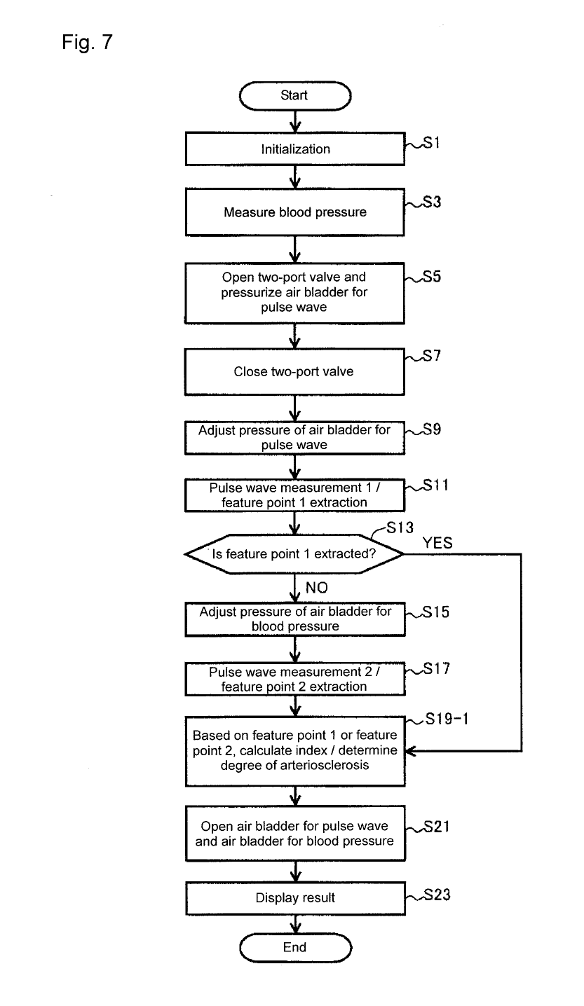

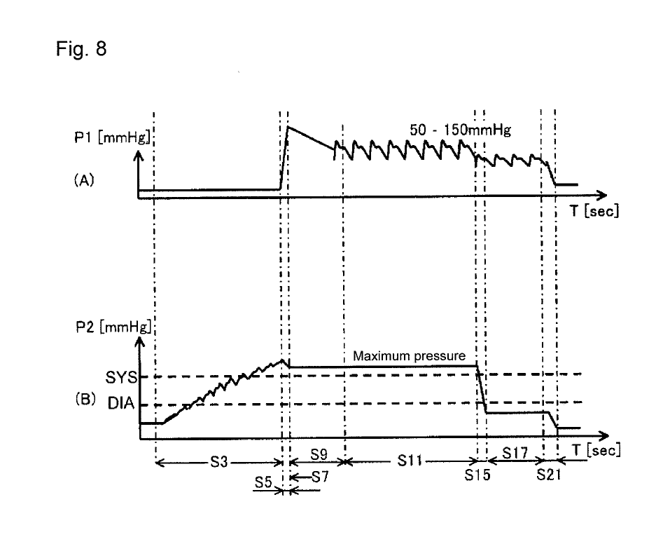

A first specific example of an operation performed by the measurement device 1A will be described with reference to FIG. 7. The first specific example is an example of a measuring operation when calculation is performed by a first arithmetic algorithm. The operation shown in FIG. 7 is started when a subject or the like presses down a measurement button on the operation unit 3 of the base body 2. This operation is achieved by the CPU 40. The CPU 40 reads a program stored in the memory 41 and controls each unit as shown in FIG. 6. In FIG. 8, a portion (A) illustrates a temporal change of a pressure P1 in the air bladder 13B, and a portion (B) illustrates a temporal change of a pressure P2 in the air bladder 13A. In the portions (A) and (B) of FIG. 8, S3 to S17 attached to temporal axes correspond to respective operations of the measuring operation performed by the measurement device 1A.

Referring to FIG. 7, when the operation starts, first, the CPU 40 performs initialization of each unit (step S1). Subsequently, the CPU 40 starts to pressurize the air bladder 13A by outputting a control signal to the air system 20A, and measures a blood pressure during the pressurizing process (step S3). The measurement of the blood pressure in step S3 may be performed by a measurement method used in an ordinary sphygmomanometer. More specifically, the CPU 40 measures a systolic blood pressure (SYS) and a diastolic blood pressure (DIA) based on a pressure signal obtained from the pressure sensor 23A. In the example of (B) of FIG. 8, the pressure P2 in the air bladder 13A increases to a pressure more than the systolic blood pressure in a period of step S3. As shown in (A) of FIG. 8, the pressure P1 in the air bladder 13B is maintained at an initial pressure in the above period.

When measuring of the blood pressure is finished in step S3, the CPU 40 outputs a control signal to the drive circuit 53 to open both of the valves of the two-port valve 51 on the side of the air bladder 13A and on the side of the air bladder 13B (step S5). Thereby, a portion of the air in the air bladder 13A moves to the air bladder 13B to pressurize the air bladder 13B.

In the example of (A) of FIG. 8, the valves of the two-port valve 51 are opened in step S5, whereby a portion of the air in the air bladder 13A moves to the air bladder 13B, and the pressure P2 is reduced. At the same time, as shown in (B) of FIG. 8, the pressure P1 in the air bladder 13B rapidly increases. Then, when the pressure P1 and the pressure P2 become the same, that is, when the internal pressures of the air bladders 13A, 13B are balanced, the moving of air from the air bladder 13A to the air bladder 13B is finished. At this point, the CPU 40 outputs a control signal to the drive circuit 53 and closes the valves of the two-port valve 51 that were opened in step S5 (step S7). In (A) and (B) of FIG. 8, it is shown that the pressure P1 and the pressure P2 are the same in step S7.

Thereafter, the CPU 40 outputs a control signal to the drive circuit 27B to adjust and reduce the pressure P1 in the air bladder 13B (step S9). The amount of reduction adjustment at this time according to one or more embodiments of the present invention, is about 5.5 mmHg/sec. Alternatively, the pressure P1 is reduced and adjusted to a pressure appropriate for pulse wave measurement, i.e., 50 to 150 mmHg. On the other hand, at this time, the pressure P2 of the air bladder 13A is maintained at a pressure higher than at least the systolic blood pressure, i.e., maximum pressure. Thereby, the air bladder 13A avascularizes the artery at the peripheral side of the measurement portion. This state is called the avascularized state. In other words, the avascularized state is a state in which the pressure P2 in the air bladder 13A presses the peripheral side of the measurement portion with a pressure higher than at least the systolic blood pressure. Thereafter, in the avascularized state, the CPU 40 measures the pressure P1 in the air bladder 13B based on a pressure signal given by the pressure sensor 23B and thereby measures the pulse wave, thus extracting feature points (step S11). In the example of FIG. 5, the pulse wave 1, i.e., the pulse wave during the avascularization, is measured in step S11, and features points A1 and B1 are extracted based on the pulse wave 1. In the below description, the pulse wave measured in step S11 is adopted as the pulse wave 1, and the extracted feature point is adopted as a feature point 1.

In a case where the feature point 1 is not extracted from the pulse wave 1 in step S11 (NO in step S13), the CPU 40 performs the following control. Herein, as described above, there is a possibility that in particular the point B1, i.e., the peak of the reflection wave, might not be extracted. Accordingly, the CPU 40 outputs a control signal to the drive circuit 27A to adjust and further reduce the pressure P2 in the air bladder 13A (step S15). Alternatively, the air valve 22A may be opened. In step S15, the CPU 40 adjusts and reduces the pressure P2 to a pressure less than at least the systolic blood pressure, i.e., about 55 mmHg, for example. Thereby, the air bladder 13A attains a state in which the artery is not avascularized or an avascularized state having a pressure weaker than that of step S11. These states are called the non-avascularized state. In other words, the non-avascularized state is a state in which the pressure P2 in the air bladder 13A presses the peripheral side of the measurement portion with a pressure lower than at least the systolic blood pressure. In the example of (B) of FIG. 8, the pressure P2 in the air bladder 13A decreases to a pressure less than the systolic blood pressure in a period of step S15. Thereafter, in the non-avascularized state, the CPU 40 measures, in the same manner as step S11, the pressure P1 in the air bladder 13B based on a pressure signal given by the pressure sensor 23B and thereby measures the pulse wave, thus extracting feature points (step S17). In the example of FIG. 5, the pulse wave 2, i.e., the pulse wave during the non-avascularization, is measured in step S17, and features points A2 and B2 are extracted based on the pulse wave 2. In the below description, the pulse wave measured in step S17 is adopted as the pulse wave 2, and the extracted feature point is adopted as a feature point 2. It should be noted that in step S17, the CPU 40 may extract, from the pulse wave 2, only the feature points that have not been extracted in step S11. In step S11, there is a possibility that the point B1 might not be extracted from the pulse wave 1. In this case, in step S17, the CPU 40 may extract only the point B2 as the feature point 2 from the pulse wave 2. Steps S15, S17 are skipped when all the feature points 1 are extracted in step S11 (YES in step S13).

When the feature point 1 is extracted in step S11, the CPU 40 calculates the above index from the feature point 1. When the feature point 1 is not extracted in step S11, and the feature point 2 is extracted in step S17, the CPU 40 calculates the index from the feature point 2. Then, the CPU determines the degree of arteriosclerosis based on the index (step S19-1). Thereafter, the CPU 40 outputs control signals to the drive circuits 27A, 27B to open the air valves 22A, 20B, thereby releasing the pressures of the air bladders 13A, 13B to the atmospheric pressure (step S21). In the example of (A) and (B) of FIG. 8, the pressures P1, P2 in the air bladders 13A, 13B rapidly decrease to the atmospheric pressure in a period of step S21.

Thereafter, the CPU 40 displays the measurement results upon performing processes for causing the display unit 4 on the base body 2 to display the calculated systolic blood pressure (SYS), the diastolic blood pressure (DIA), the measurement results such as the measured pulse waves, and the determination result of the degree of arteriosclerosis (step S23).

In the measuring operation according to the first specific example, when the feature point 2 is not extracted in step S17, the internal pressure P1 of the air bladder 13B may be adjusted and reduced. That is, the internal pressure P1 may be repeatedly adjusted and reduced until all the feature points are extracted. Further, at this time, the measuring operation may be terminated when the internal pressure P1 has reached a predetermined pressure, or the measuring operation may be terminated when the internal pressure P1 has been reduced and adjusted for a predetermined number of times.

The measurement device 1A achieves the measuring operation according to the first specific example as shown in FIG. 7, thus measuring the pulse wave in the non-avascularized state (pulse wave 2), in a case where it is difficult to find the feature points and the feature points are not extracted from the pulse wave 1 of FIG. 5 measured in the avascularized state. Especially when the peripheral side is avascularized, most of the reflection wave from the peripheral side is shielded, which may prevent extraction of the feature point (B1 point) corresponding to the peak of the reflection wave. However, in such a case, the measurement device 1A measures the pulse wave at the peripheral side in the non-avascularized state, thus easily extracting the feature point (B2 point) corresponding to the peak of the reflection wave in particular. Therefore, the index can be accurately calculated, and the index useful for determining the degree of arteriosclerosis can be obtained.

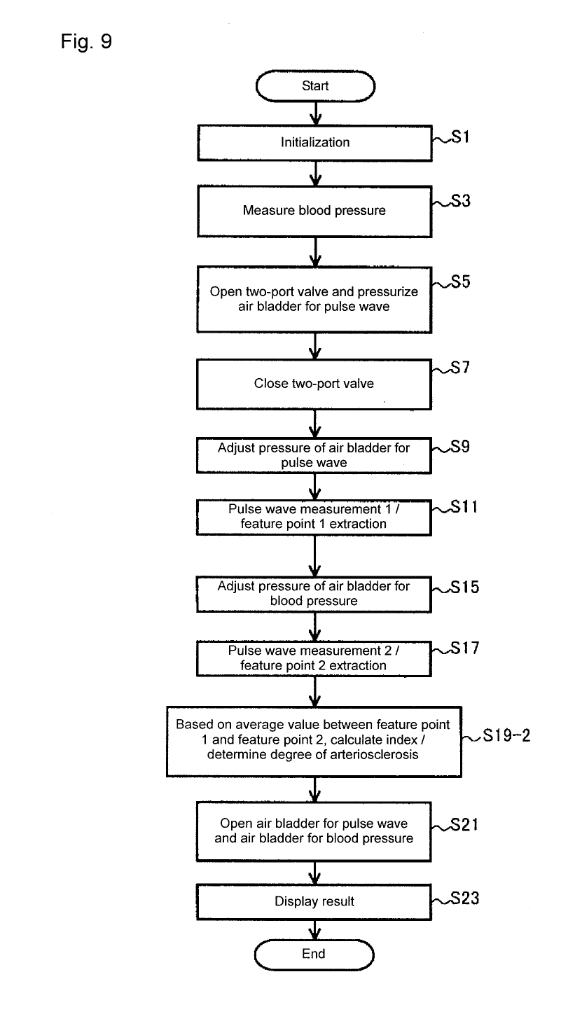

A second specific example of the operation performed by the measurement device 1A will be described with reference to FIG. 9. The second specific example is an example of a measuring operation when calculation is performed according to a second arithmetic algorithm. The operation shown in FIG. 9 is also started when a subject or the like presses down the measurement button on the operation unit 3 of the base body 2. This operation is achieved by the CPU 40. The CPU 40 reads a program stored in the memory 41 and controls each unit as shown in FIG. 6. In FIG. 9, the same measuring operation as that of the first specific example shown in the flowchart of FIG. 7 is denoted with the same step number. Accordingly, S3 to S17 attached to the temporal axes of (A) and (B) of FIG. 8 correspond to each operation of the measuring operation shown in FIG. 9.

Referring to FIG. 9, in the measuring operation according to the second specific example, the pulse wave 1 is measured in the avascularized state in step S11, and the feature point 1 is extracted from the pulse wave 1. Thereafter, the operation of step S15 is performed to further reduce and adjust the pressure P1 in the air bladder 13B. Then, in step S17, the pulse wave 2 is measured in the non-avascularized state, and the feature point 2 is extracted from the pulse wave 2. Subsequently, in the measuring operation according to the second specific example, different from the measuring operation according to the first specific example, the CPU 40 calculates an average value between the feature point 1 extracted in step S11 and the feature point 2 extracted in step S17, and calculates the index from the average value, thereby determining the degree of arteriosclerosis (step S19-2). In other words, when Tpp is calculated as the index, the CPU 40 calculates an average between an emerging time of the point A1 extracted from the pulse wave 1 in step S11 and an emerging time of the point A2 extracted from the pulse wave 2 in step S17 and an average between an emerging time of the point B1 extracted from the pulse wave 1 in step S11 and an emerging time of the point B2 extracted from the pulse wave 2 in step S17, and the CPU 40 obtains Tpp by calculating a difference therebetween. When AI is calculated as the index, the CPU 40 calculates an average between an amplitude of the point A1 extracted from the pulse wave 1 in step S11 and an amplitude of the point A2 extracted from the pulse wave 2 in step S17 and an average between an amplitude of the point B1 extracted from the pulse wave 1 in step S11 and an amplitude of the point B2 extracted from the pulse wave 2 in step S17, and the CPU 40 obtains AI according to a ratio therebetween. Thereafter, the operation of steps S21, S23 is performed.

When the measurement device 1A achieves the measuring operation according to the second specific example as shown in FIG. 9, the index is calculated using an average between the feature points (A1, B1) extracted from the pulse wave (pulse wave 1) measured in the avascularized state and the feature points (A2, B2) extracted from the pulse wave (pulse wave 2) measured in the non-avascularized state. Therefore, the index can be accurately calculated, and the index useful for determining the degree of arteriosclerosis can be obtained.

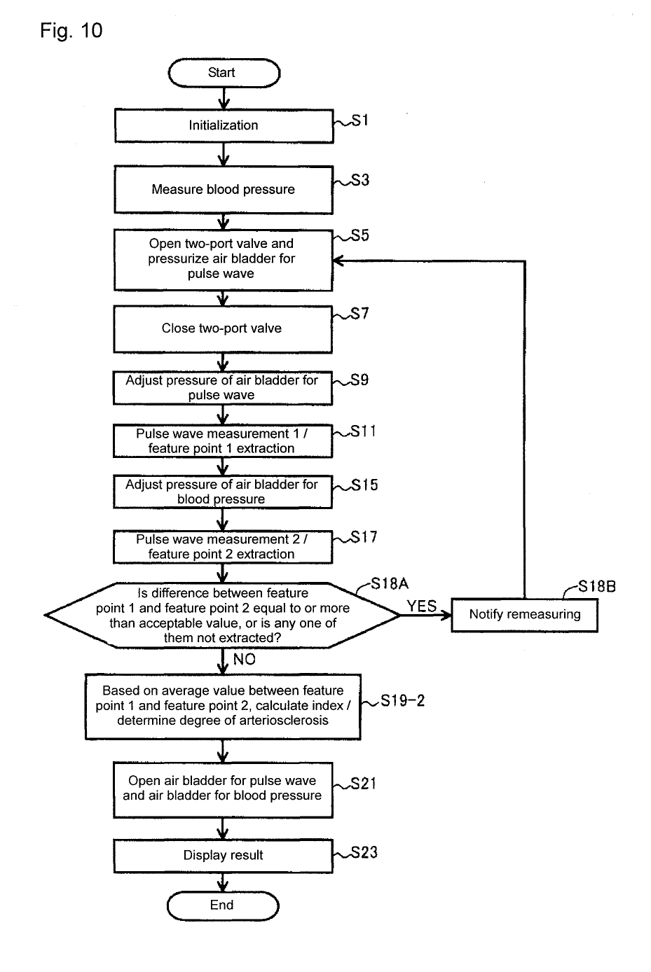

A third specific example of the operation performed by the measurement device 1A will be described with reference to FIG. 10. The third specific example is an example of a measuring operation when calculation is performed according to a third arithmetic algorithm. The operation shown in FIG. 10 is also started when a subject or the like presses down the measurement button on the operation unit 3 of the base body 2. This operation is achieved by the CPU 40. The CPU 40 reads a program stored in the memory 41 and controls each unit as shown in FIG. 6. In FIG. 10, the same measuring operation as that of the first specific example shown in the flowchart of FIG. 7 and that of the second specific example shown in the flowchart of FIG. 9 is denoted with the same step number. Accordingly, S3 to S17 attached to the temporal axes of (A) and (B) of FIG. 8 correspond to each operation of the measuring operation shown in FIG. 10.

Referring to FIG. 10, in the measuring operation according to the third specific example, the pulse wave 1 is measured in the avascularized state in step S11, and the feature point 1 is extracted from the pulse wave 1. Thereafter, the operation of step S15 is performed to further reduce and adjust the pressure P1 in the air bladder 13B. Then, in step S17, the pulse wave 2 is measured in the non-avascularized state, and the feature point 2 is extracted from the pulse wave 2. Subsequently, in the measuring operation according to the third specific example, different from the measuring operations according to the first and second specific examples, the CPU 40 compares the feature point 1 extracted in step S11 and the feature point 2 extracted in step S17, and determines whether a difference therebetween is equal to or more than an acceptable value (step S18A). More specifically, a difference between an emerging time of the point A1 extracted from the pulse wave 1 in step S11 and an emerging time of the point A2 extracted from the pulse wave 2 in step S17 and/or a difference between an emerging time of the point B1 extracted from the pulse wave 1 in step S11 and an emerging time of the point B2 extracted from the pulse wave 2 in step S17 are calculated, and determination is made as to whether the difference is equal to or more than the acceptable value. For example, an acceptable value is about 10 ms, and is stored to the CPU 40 in advance. Alternatively, the acceptable value may be registered and updated by predetermined operation (for example, an operation method known to a user such as a doctor specified in advance). As described above, the emerging time of the point A1 and the emerging time of the point A2 are considered to be substantially the same for the same subject. Likewise, the emerging time of the point B1 and the emerging time of the point B2 are considered to be substantially the same. Accordingly, when the difference between these emerging times is equal to or more than the acceptable value, it is considered that either of the pulse waves is not correctly measured or the feature points are not correctly extracted.

Accordingly, in a case where, in step S18A, the difference between the feature point 1 and the feature point 2 is determined to be equal to or more than the acceptable value, or one of the feature point 1 and the feature point 2 is not extracted (NO in step S18A), the CPU 40 performs an operation for causing the display unit 4 to display a screen for notifying remeasuring. Then, after the CPU 40 notifies remeasuring (step S18B), the CPU 40 causes the measuring operation to return to step S5, and opens the two-port valve 51 again.

In a case where the feature point 1 is extracted in step S11, the feature point 2 is extracted in step S17, and the difference therebetween is within the acceptable value (YES in step S18A), then the CPU 40 calculates an average value between the feature point 1 extracted in step S11 and the feature point 2 extracted in step S17, and calculates the index from the average value, thereby determining the degree of arteriosclerosis (step S19-2), in the same manner as the measuring operation according to the second specific example. Alternatively, the index may be calculated using one of the feature point 1 extracted in step S11 and the feature point 2 extracted in step S17, or the index may be calculated using the feature point 1 extracted from the pulse wave 1 measured in the avascularized state in step S11.

The measurement device 1A performs the measuring operation according to the third specific example as shown in FIG. 10. Accordingly, remeasuring is performed when a difference between the feature points (point A1, point B1) extracted from the pulse wave (pulse wave 1) measured in the avascularized state and the feature points (point A2, point B2) extracted from the pulse wave (pulse wave 2) measured in the non-avascularized state is equal to or more than the acceptable value. Therefore, the index can be accurately calculated, and the index useful for determining the degree of arteriosclerosis can be obtained.

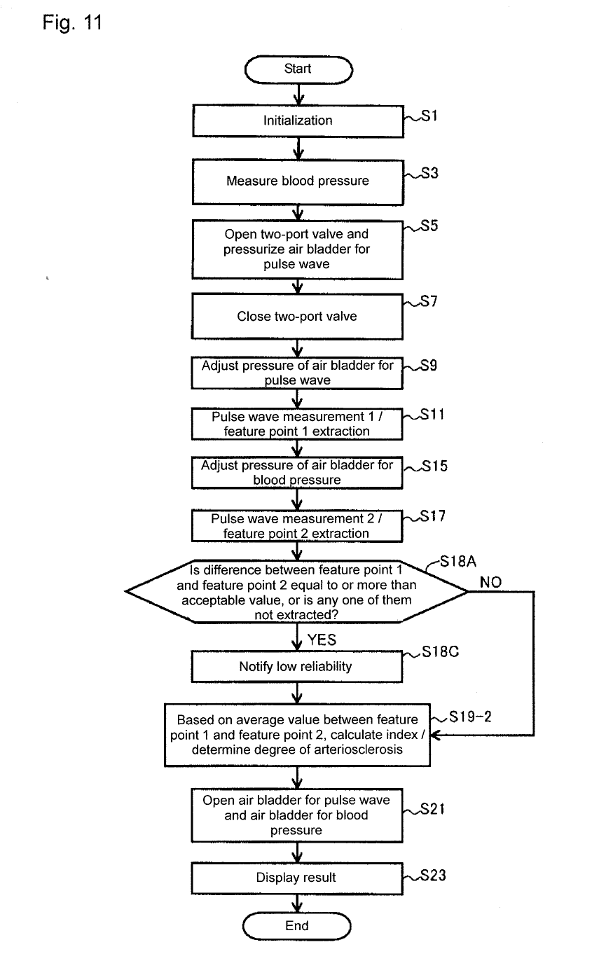

The fourth specific example of the operation performed by the measurement device 1A will be described with reference to FIG. 11. The fourth specific example is an example of a measuring operation when calculation is performed according to a fourth arithmetic algorithm. The operation shown in FIG. 11 is also started when a subject or the like presses down the measurement button on the operation unit 3 of the base body 2. This operation is achieved by the CPU 40. The CPU 40 reads a program stored in the memory 41 and controls each unit as shown in FIG. 6. In FIG. 11, the same measuring operation as the measuring operation of the first specific example shown in the flowchart of FIG. 7, the measuring operation of the second specific example shown in the flowchart of FIG. 9, and the measuring operation of the third specific example shown in the flowchart of FIG. 10 is denoted with the same step number. Accordingly, S3 to S17 attached to the temporal axes of (A) and (B) of FIG. 8 correspond to each operation of the measuring operation shown in FIG. 11.

Referring to FIG. 11, in the measuring operation according to the fourth specific example, in a case where in step S18A, the difference between the feature point 1 and the feature point 2 is determined to be equal to or more than the acceptable value, or one of the feature point 1 and the feature point 2 is not extracted (NO in step S18A), the CPU 40 performs processing for causing the display unit 4 to display a screen for notifying that the determination result has a low reliability. Then, the CPU 40 performs the measuring operation after notifying to that effect (step S18C). In the same manner as the measuring operation according to the second specific example and the measuring operation according to the third specific example, the CPU 40 calculates an average value between the feature point 1 extracted in step S11 and the feature point 2 extracted in step S17, and calculates the index from the average value, thereby determining the degree of arteriosclerosis (step S19-2).

The measurement device 1A achieves the measuring operation according to the fourth specific example as shown in FIG. 11. Accordingly, even when a difference between the feature points (point A1, point B1) extracted from the pulse wave (pulse wave 1) measured in the avascularized state and the feature points (point A2, point B2) extracted from the pulse wave (pulse wave 2) measured in the non-avascularized state is equal to or more than the acceptable value, the measurement device 1A notifies that the determination result has a low reliability and calculates the index using these feature points. Therefore, although the calculated index has a lower reliability than the index obtained from the measuring operation according to the third specific example, remeasuring is not performed, and the index is calculated from one measuring operation, whereby the degree of arteriosclerosis can be determined in a shorter time.

Further, as described above, in the measurement device 1A, the air bladder 13A and the air bladder 13B are connected via the two-port valve 51. Then, when measuring of the blood pressure is finished in step S3, the two-port valve 51 is opened in step S5, whereby the air in the air bladder 13A is moved to the air bladder 13B. When the two-port valve 51 is opened, the air in the air bladder 13A rapidly blows into the air bladder 13B in order to eliminate a pressure difference. Therefore, a time needed to blow air into the air bladder 13B using a pump can be greatly reduced, and the overall measuring time can be reduced. This can reduce the strain imposed on the subject. In general, when it takes a long time to perform measurement, an artery is pressed for a long time, which stimulates sympathetic nerves and may deteriorate the characteristics of blood vessels. In contrast, an artery is pressed for a shorter time, when the measurement is performed in a shorter time. In general, body movement is more likely to occur as the measuring takes a longer time. However, when the measurement is performed in a shorter time, the body movement is less likely to occur. Therefore, blood pressure information such as pulse waves can be measured with higher accuracy. In addition, the accuracy of the index of arteriosclerosis obtained from the measurement result can also be improved.

As shown in FIG. 6, a mechanism for blowing air into the air bladder 13B (air pump, air pump drive circuit) may not be arranged. This can contribute to making the apparatus smaller, lighter, and inexpensive.

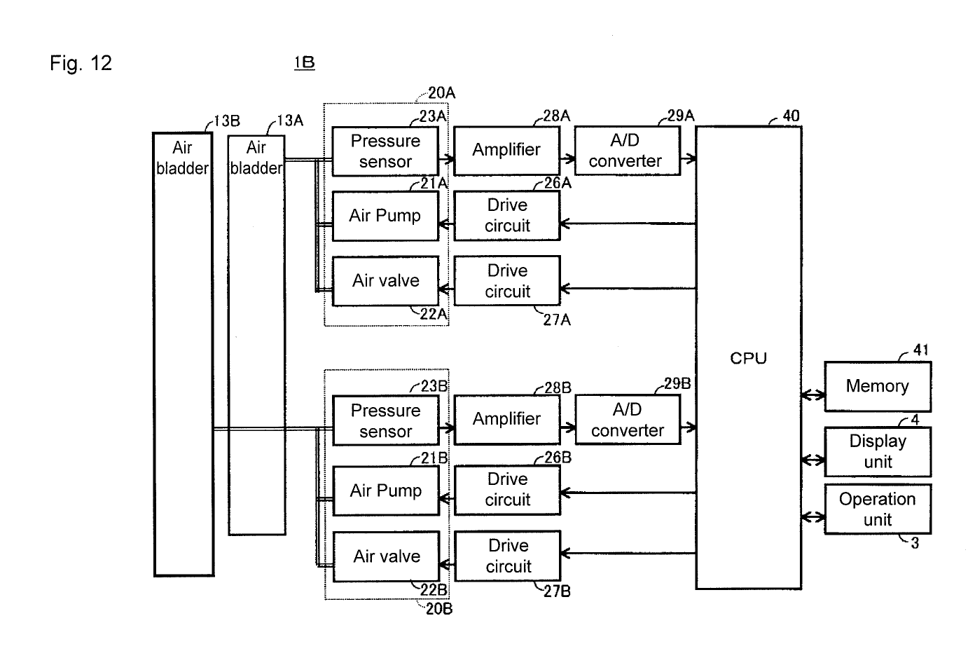

However, the above measuring operation can be performed not only by the measurement device having the configuration as shown in FIG. 6 but also by the measurement device having an ordinary configuration as shown in FIG. 12. Accordingly, the second embodiment will be described. In the second embodiment, the measuring operation is performed by the measurement device 1B having the configuration as shown in FIG. 12.

### Second Embodiment

The measurement device 1B is generally the same as the measurement device 1A shown in FIG. 1. Referring to FIG. 12, in the measurement device 1B, an air system 20B includes an air pump 21B, and the measurement device 1B includes a drive circuit 26B for driving the air pump 21B, in place of the two-port valve 51 and the drive circuit 53 of the configuration of the measurement device 1A as shown in FIG. 6. The air pump 21 B is driven by the drive circuit 26B receiving an instruction from the CPU 40, and blows compressed gas into the air bladder 13B.

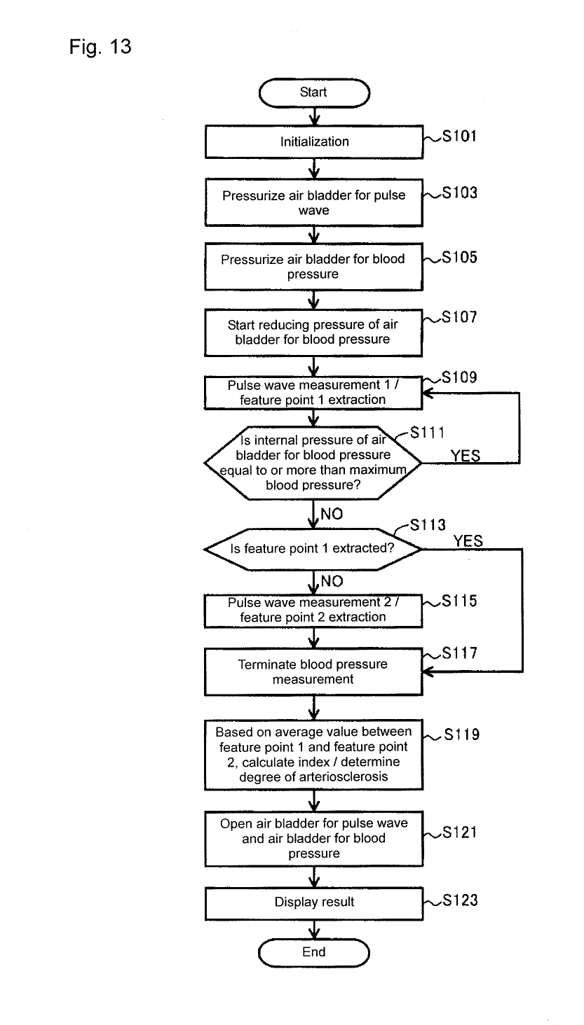

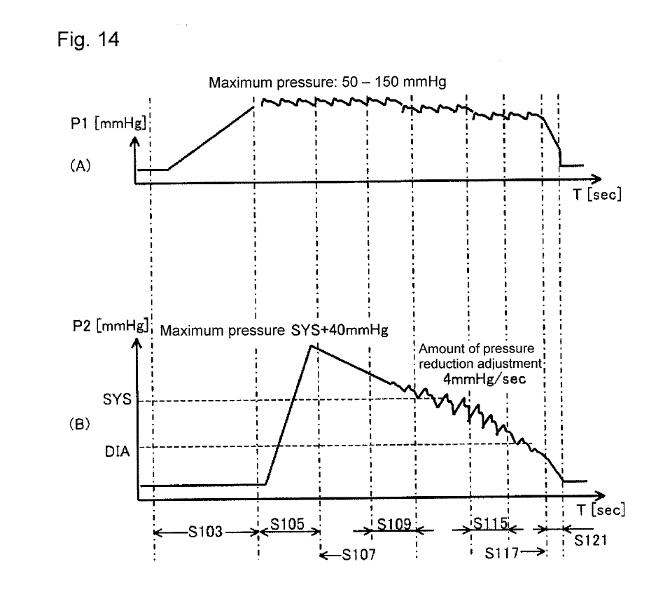

A first specific example of an operation of the measurement device 1B will be described with reference to FIG. 13. The first specific example represents a measuring operation when calculation is performed according to the first arithmetic algorithm described in the first embodiment. The operation shown in FIG. 13 is started when a subject or the like presses down the measurement button on the operation unit 3 of the base body 2. This operation is achieved by the CPU 40. The CPU 40 reads a program stored in the memory 41 and controls each unit as shown in FIG. 12. In FIG. 14, a portion (A) represents a temporal change of the pressure P1 in the air bladder 13B, and a portion (B) represents a temporal change of the pressure P2 in the air bladder 13A. In the portions (A) and (B) of FIG. 14, S103 to S121 attached to temporal axes correspond to respective operations of the measuring operation performed by the measurement device 1B.

Referring to FIG. 13, when the operation starts, the CPU 40 performs initialization of each unit (step S101). Subsequently, the CPU 40 outputs a control signal to the air system 20B, and pressurizes the air bladder 13B to a predetermined pressure (step S103). In the example of (A) of FIG. 14, the pressure P1 in the air bladder 13B increases within a period of step S103. Then, the pressure P1 is thereafter maintained. In step S103, the pressure P1 is increased so that the pressure P1 attains a pressure appropriate for pulse wave measurement, i.e., 50 to 150 mmHg. When the pressure P1 attains the predetermined pressure, the CPU 40 outputs a control signal to the air system 20A, increases the pressure P2 of the air bladder 13A to a predetermined pressure, and causes the air bladder 13A to pressurize the peripheral side of the measurement portion (step S105). In the example of (B) of FIG. 14, the pressure P2 in the air bladder 13A increases within a period of step S105. In step S105, the CPU 40 increases the pressure P2 until the pressure P2 attains a pressure higher than the general systolic blood pressure value. According to one or more embodiments of the present invention, the pressure P2 is increased to about the systolic blood pressure value +40 mmHg. Therefore, the air bladder 13A avascularizes an artery. Thereafter, the CPU 40 outputs a control signal to the air system 20A, and starts reducing the pressure P2 in the air bladder 13A (step S107). In this case, the amount of pressure reduction adjustment is about 4 mmHg/sec, and the pressure P2 is gradually reduced.

While the pressure P2 in the air bladder 13A changes from the maximum pressure to the systolic blood pressure during the pressure reduction process of the pressure P2 in the air bladder 13A (YES in step S111), namely, in the avascularized state, the CPU 40 measures a pulse wave by measuring the pressure P1 in the air bladder 13B based on a pressure signal given by the pressure sensor 23B, thereby extracting a feature point (step S109). In a period shown in step S109 in (A) and (B) of FIG. 14, the pulse wave is measured, and the feature point is extracted. In the example of FIG. 5, the pulse wave 1, i.e., the pulse wave during the avascularization, is measured in step S109, and features points A1 and B1 are extracted based on the pulse wave 1. It should be noted that, for the sake of the below description, the pulse wave measured in step S109 will be referred to as the pulse wave 1, and the extracted feature point will be referred to as the feature point 1.

In a case where the feature point 1 is not extracted from the pulse wave 1 (NO in step S113) while the pressure P2 in the air bladder 13A changes to the systolic blood pressure value during the pressure reduction process of the pressure P2 in the air bladder 13A, the CPU 40 measures a pulse wave by measuring the pressure P1 in the air bladder 13B based on a pressure signal given by the pressure sensor 23B and thereby extracts a feature point while the pressure P2 in the air bladder 13A is less than the systolic blood pressure value during the pressure reduction process of the pressure P2 in the air bladder 13A, namely, in the non-avascularized state (step S115). In a period shown in step S115 in (A) and (B) of FIG. 14, the pulse wave is measured, and the feature point is extracted. In the example of FIG. 5, the pulse wave 2, i.e., the pulse wave during the non-avascularization, is measured in step S115, and features points A2 and B2 are extracted based on the pulse wave 2. For the sake of the below description, the pulse wave measured in step S115 will be referred to as the pulse wave 2, and the extracted feature point will be referred to as the feature point 2. Step S115 is skipped when all the feature points 1 are extracted in step S109 (YES in step S113).

In the pressure reduction process since around a time when the internal pressure of the air bladder 13A reaches the systolic blood pressure value after step S109, the CPU 40 measures the above pulse wave as well as the blood pressure. The measurement of the blood pressure may be performed by a measurement method used in an ordinary sphygmomanometer. More specifically, the CPU 40 calculates a systolic blood pressure (SYS) and a diastolic blood pressure (DIA) based on a pressure signal obtained from the pressure sensor 23A. The CPU 40 terminates the measuring of the blood pressure when the systolic blood pressure value and the diastolic blood pressure value are calculated or when the internal pressure of the air bladder 13A becomes lower than the diastolic blood pressure value (step S117).

When the feature point 1 is extracted in step S109, the CPU 40 calculates the index from the feature point 1. When the feature point 1 is not extracted in step S109, and the feature point 2 is extracted in step S115, the CPU 40 calculates the index from the feature point 2. Then, the CPU determines the degree of arteriosclerosis based on the index (step S119). Thereafter, the CPU 40 outputs control signals to the drive circuits 27A, 27B to open the air valves 22A, 20B, thereby releasing the pressures in the air bladders 13A, 13B to atmospheric pressure (step S121). In the example of (A) and (B) of FIG. 14, the pressures P1, P2 in the air bladders 13A, 13B rapidly decrease to the atmospheric pressure in a period of step S121.

Thereafter, the CPU 40 displays the measurement results upon performing processes for causing the display unit 4 on the base body 2 to display the calculated systolic blood pressure (SYS), the diastolic blood pressure (DIA), the measurement results such as the measured pulse waves, and the determination result of the degree of arteriosclerosis (step S123).

The measurement device 1B achieves the measuring operation according to the first specific example as shown in FIG. 13, thus measuring the pulse wave in the non-avascularized state (pulse wave 2), in a case where it is difficult to find the feature points and the feature points are not extracted from the pulse wave 1 of FIG. 5 measured in the avascularized state. Especially when the peripheral side is avascularized, most of the reflection wave from the peripheral side is shielded, which may prevent extraction of the feature point (B1 point) corresponding to the peak of the reflection wave. However, in such a case, the measurement device 1B measures the pulse wave at the peripheral side in the non-avascularized state, thus easily extracting the feature point (B2 point) corresponding to the peak of the reflection wave in particular. Therefore, the index can be accurately calculated, and the index useful for determining the degree of arteriosclerosis can be obtained.

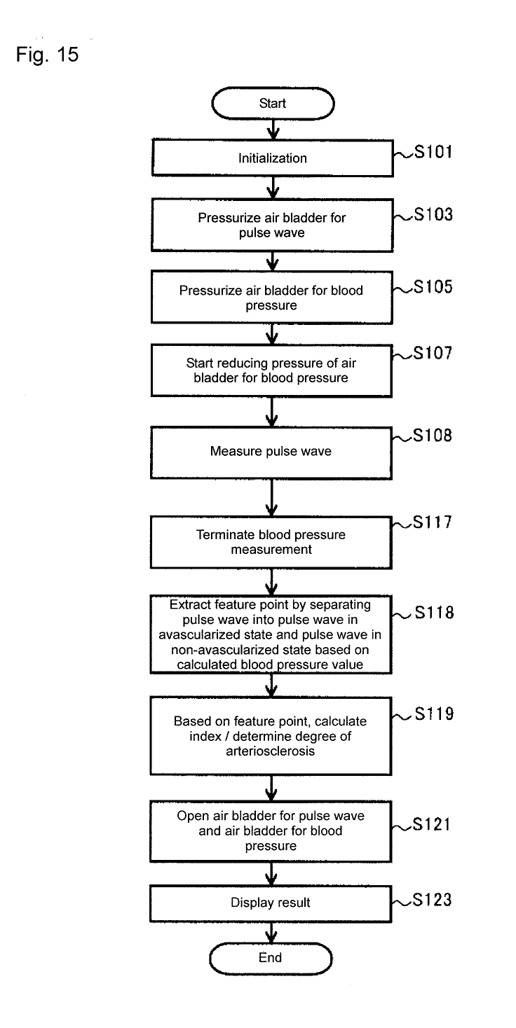

A second specific example of the operation performed by the measurement device 1B will be described with reference to FIG. 15. The second specific example represents a measuring operation when calculation is performed according to the second arithmetic algorithm described in the first embodiment. The operation shown in FIG. 15 is also started when a subject or the like presses down the measurement button on the operation unit 3 of the base body 2. This operation is achieved by the CPU 40. The CPU 40 reads a program stored in the memory 41 and controls each unit as shown in FIG. 12. In FIG. 15, the same measuring operation as that of the first specific example shown in the flowchart of FIG. 13 is denoted with the same step number.

Referring to FIG. 15, the measuring operation according to the second specific example is as follows. When the pressure P2 in the air bladder 13A begins to be reduced in step S107, the CPU 40 measures a pulse wave by measuring the pressure P1 in the air bladder 13B based on a pressure signal given by the pressure sensor 23B in the pressure reduction process (step S108). At this time, the CPU 40 measures the pressure P2 in the air bladder 13A based on a pressure signal obtained from the pressure sensor 23A, and stores the measured pulse wave as well as the pressure P2 in the air bladder 13A during the measuring operation to a predetermined region of the memory 41. In the example of (A), (B) of FIG. 14, step S108 corresponds to periods of steps S109, S115.

When the measurement of the pulse wave in step S108 is finished, the CPU 40 obtains the systolic blood pressure (SYS). The systolic blood pressure (SYS) may be obtained by performing calculation based on the pressure signal obtained from the pressure sensor 23A. Alternatively, the systolic blood pressure (SYS) may be obtained by receiving an input with predetermined buttons and the like on the operation unit 3. Alternatively, the systolic blood pressure (SYS) may be stored to the memory 41 as a general value in advance and may be obtained from the memory 41. The CPU 40 compares the pressure P2 in the air bladder 13A during the measurement process stored in association with the measured pulse wave and the obtained systolic blood pressure, thereby determining whether the measured pulse wave is measured in the avascularized state or measured in the non-avascularized state. In other words, the systolic blood pressure is used as a threshold value for determining whether it is measured in the avascularized state or in the non-avascularized state. It should be noted that the obtained systolic blood pressure may be a case where the pressure P2 in the air bladder 13A is lower than the diastolic blood pressure (DIA) lower than the systolic blood pressure. In such a case, the diastolic blood pressure is also used as the threshold value for comparison with the diastolic blood pressure, whereby the measured pulse wave is determined to be measured in the non-avascularized state.

Then, the CPU 40 extracts the feature point from the measured pulse wave (step S118), and calculates the index from the feature point, thereby determining the degree of arteriosclerosis (step S119). In this case, when the points A1 and B1, i.e., the feature points, are extracted from the pulse wave 1 measured in the avascularized state, these may be used to calculate the index in the same manner as the above-described calculation performed according to the first arithmetic algorithm. Alternatively, in the same manner as the calculation performed according to the second arithmetic algorithm, the index may be calculated using respective averages between the points A1 and B1, i.e., the feature points, extracted from the pulse wave 1 measured in the avascularized state and between the points A2 and B2, i.e., the feature points, extracted from the pulse wave 2 measured in the non-avascularized state. Alternatively, in the same manner as the calculation performed according to the third arithmetic algorithm, when respective differences between the points A1 and B1, i.e., the feature points, extracted from the pulse wave 1 measured in the avascularized state and between the points A2 and B2, i.e., the feature points, extracted from the pulse wave 2 measured in the non-avascularized state are within the acceptable value, the index may be calculated using either of the feature points or the average value thereof. Hereinafter, the operation of steps S121, S123 is performed.

The measurement device 1B achieves the measuring operation according to the second specific example as shown in FIG. 15. Accordingly, it is not necessary to adjust the pressure P2 in the air bladder 13A to a predetermined pressure so that the peripheral side of the measurement portion is in the avascularized state or the non-avascularized state. In other words, for example, the pressure P2 is reduced with a constant pressure reduction adjustment amount such as about 4 mmHg/sec, and determination can be made as to whether the pulse wave measured during the pressure reduction process is the pulse wave (pulse wave 1) in the avascularized state or the pulse wave (pulse wave 2) in the non-avascularized state by comparing the pressure P2 during the measurement and the blood pressure value. Therefore, the index can be accurately calculated without any complicated control, and the index useful for determining the degree of arteriosclerosis can be obtained. Further, since it is not necessary to adjust the pressure P2, the measuring operation can be performed in a shorter time.

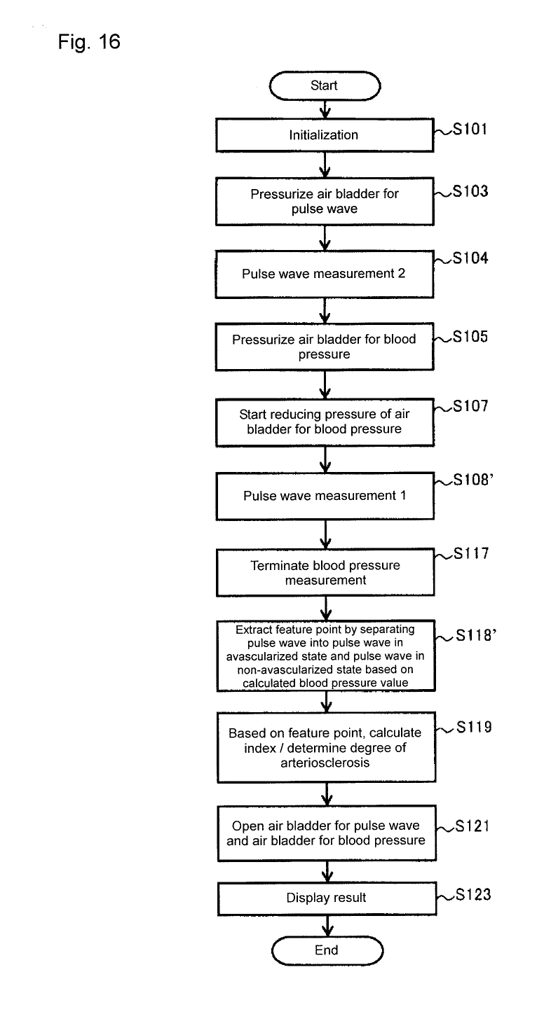

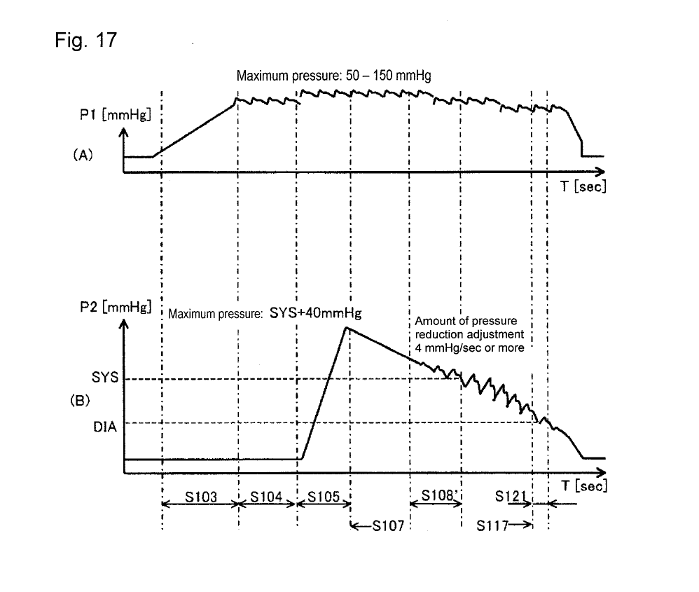

As a modification of the measuring operation according to the second specific example, the measurement device 1B can perform a measuring operation as shown in FIG. 16. The modification of the measuring operation according to the second specific example represents a modification of the measuring operation when calculation is performed according to the first arithmetic algorithm described in the second embodiment. The operation shown in FIG. 16 is also started when a subject or the like presses down the measurement button on the operation unit 3 of the base body 2. This operation is achieved by the CPU 40. The CPU 40 reads a program stored in the memory 41 and controls each unit as shown in FIG. 12. In FIG. 17, a portion (A) illustrates a temporal change of the pressure P1 in the air bladder 13B, and a portion (B) illustrates a temporal change of the pressure P2 in the air bladder 13A. In the portions (A) and (B) of FIG. 17, S103 to S121 attached to temporal axes correspond to respective operations of the measuring operation performed by the measurement device 1B.

Referring to FIG. 16, in the modification of the measuring operation according to the second specific example, the CPU 40 measures a pulse wave by measuring the pressure P1 in the air bladder 13B based on a pressure signal given by the pressure sensor 23B (step S104) when the pressure P1 in the air bladder 13B is in a pressurized state so as to attain a pressure suitable for pulse wave measurement, i.e., a range of 50 to 150 mmHg, in step S103, but before the air bladder 13A pressurizes the peripheral side of the measurement portion in subsequent step S105, namely, in the non-avascularized state. The pulse wave measured in step S105 is a pulse wave in the non-avascularized state as described above. In the description, the measured pulse wave is referred to as the pulse wave 2. In the example of (A) and (B) of FIG. 17, the pulse wave 2 is measured in a period of step S104. As shown in (B) of FIG. 17, the pressure P2 in the air bladder 13A is not pressurized and is maintained at an initial pressure in the period of step S104.

Thereafter, the CPU 40 outputs a control signal to the air system 20A, and increases the pressure P2 in the air bladder 13A to a predetermined pressure, whereby the air bladder 13A pressurizes the peripheral side of the measurement portion (step S105). According to one or more embodiments of the present invention, the predetermined pressure is about the systolic blood pressure value +40 mmHg as described above. After the pressure P2 reaches the predetermined pressure, the CPU 40 outputs a control signal to the air system 20A, and starts reducing the pressure P2 in the air bladder 13A (step S107). The amount of reduction adjustment at this time according to one or more embodiments of the present invention is about 4 mmHg/sec.

During the pressure reduction process of the pressure P2 in the air bladder 13A, the CPU 40 measures a pulse wave by measuring the pressure P1 in the air bladder 13B based on a pressure signal given by the pressure sensor 23B, thereby extracting a feature point (step S108′). At this time, the CPU 40 measures the pressure P2 in the air bladder 13A based on a pressure signal obtained from the pressure sensor 23A, and stores the measured pulse wave as well as the pressure P2 in the air bladder 13A during the measuring operation to a predetermined region of the memory 41. It should be noted that the measuring operation in step S108′ is performed mainly for the purpose of measuring the pulse wave 1 in the avascularized state since the pulse wave 2 in the non-avascularized state is measured in step S104. Accordingly, the measuring operation in step S108′ is performed in a very short period compared with step S108. According to one or more embodiments of the present invention, the measuring operation in step S108′ is performed while the pressure P2 in the air bladder 13A changes from the maximum pressure to the systolic blood pressure. In the example of (A) and (B) of FIG. 17, the pulse wave is measured in a period of step S108′. The period of step S108′ corresponds to a period of step S109 in the example of (A), (B) of FIG. 14. On the other hand, as described above, step S108 corresponds to periods of steps S109, S115 in the example of (A) and (B) of FIG. 14. That is, as shown in FIG. 14 and FIG. 17, the measuring operation of step S108′ is performed in a shorter period than the measuring operation of step S108.

Thereafter, in the pressure reduction process, namely, in the pressure reduction process in which the pressure P2 in the air bladder 13A reaches the diastolic blood pressure, the CPU 40 performs only the blood pressure measurement. Accordingly, in the pressure reduction process after step S108′, the CPU 40 increases the amount of pressure reduction adjustment. The amount of reduction adjustment according to one or more embodiments of the present invention is 4 mmHg/sec or more. When the blood pressure measurement is finished (step S117), the CPU 40 compares the pressure P2 in the air bladder 13A during the measurement process stored in association with the pulse wave measured in step S108′ with the obtained systolic blood pressure (SYS) and the diastolic blood pressure (DIA), thereby determining whether the measured pulse wave is measured in the avascularized state or measured in the non-avascularized state (step S118′). Then, the CPU 40 extracts the feature point from the measured pulse wave (step S118), and calculates the index from the feature point, thereby determining the degree of arteriosclerosis (step S119). As described above, in step S104, the pulse wave 2 in the non-avascularized state is measured. Therefore, in step S118′, the CPU 40 extracts the pulse wave 1 measured in the avascularized state from among the pulse waves measured in step S108′. Hereinafter, the measuring operation of steps S119, S121, S123 is performed.

The measurement device 1B achieves the measuring operation according to the modification of the second specific example as shown in FIG. 16. Accordingly, the pressure reduction rate of the pressure P2 in the air bladder 13A can be further increased after the measurement of the pulse wave in step S108′ is finished. Therefore, the measuring operation can be performed in a shorter time.

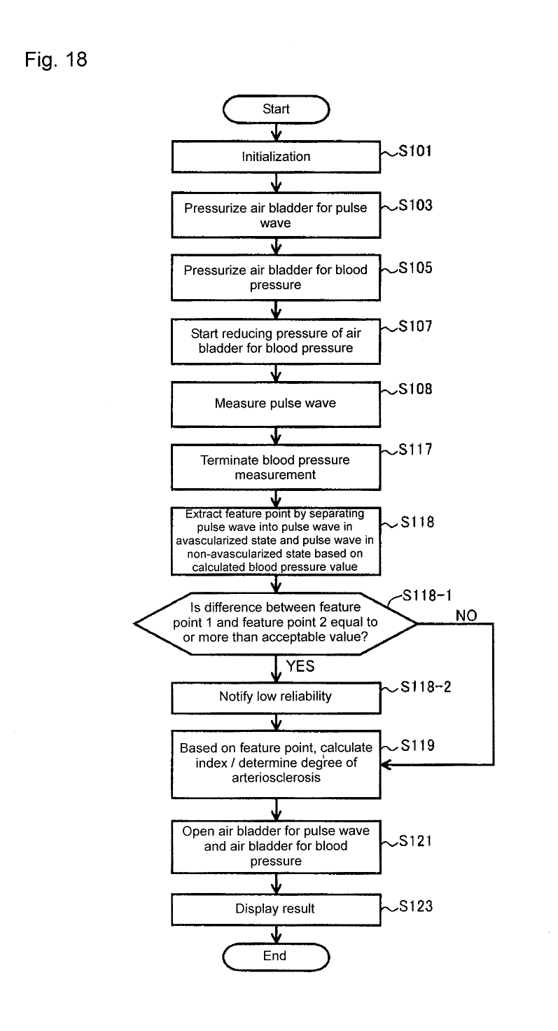

A third specific example of the operation performed by the measurement device 1B will be described with reference to FIG. 18. The third specific example represents a measuring operation when calculation is performed according to the fourth arithmetic algorithm described in the first embodiment. The operation shown in FIG. 18 is also started when a subject or the like presses down the measurement button on the operation unit 3 of the base body 2. This operation is achieved by the CPU 40. The CPU 40 reads a program stored in the memory 41 and controls each unit as shown in FIG. 12. In FIG. 18, the same measuring operation as the measuring operation of the first specific example shown in the flowchart of FIG. 13 and the measuring operation of the second specific example shown in the flowchart of FIG. 15 is denoted with the same step number.

Referring to FIG. 18, in the measuring operation according to the third specific example, the CPU 40 measures the pulse wave during the pressure reduction process of the pressure P2 in the air bladder 13A, and stores the measured pulse wave as well as the pressure P2 in the air bladder 13A during the measuring operation to a predetermined region of the memory 41, in the same manner as step S108. Then, the CPU 40 compares the pressure P2 during the measurement process with the obtained systolic blood pressure (SYS) and the diastolic blood pressure (DIA), thereby determining whether the measured pulse wave is measured in the avascularized state or measured in the non-avascularized state, in the same manner as step S109. Then, the feature point is extracted from the measured pulse wave (step S118). Further, in the measuring operation according to the third specific example, the CPU 40 compares the feature point 1 extracted from the pulse wave measured in the avascularized state and the feature point 2 extracted from the pulse wave measured in the non-avascularized state, and determines whether a difference therebetween is equal to or more than an acceptable value (step S118-1), in the same manner as step S18A. In a case where in step S118-1, the difference between the feature point 1 and the feature point 2 is determined to be equal to or more than the acceptable value (NO in step S118-1), the CPU 40 performs processing for causing the display unit 4 to display a screen for notifying that the determination result has a low reliability in the same manner as step S18C. Then, the CPU 40 performs the measuring operation after notifying to that effect (step S118-2). Then, the CPU 40 calculates the index from the extracted feature point, thereby determining the degree of arteriosclerosis, in the same manner as the measuring operation according to the second specific example.

The measurement device 1B achieves the measuring operation according to the third specific example as shown in FIG. 18. Accordingly, even when a difference between the feature points (point A1, point B1) extracted from the pulse wave (pulse wave 1) measured in the avascularized state and the feature points (point A2, point B2) extracted from the pulse wave (pulse wave 2) measured in the non-avascularized state is equal to or more than the acceptable value, the measurement device 1B notifies that the determination result has a low reliability and calculates the index using these feature points. Therefore, remeasuring is not performed, and the index is calculated from one measuring operation, whereby the degree of arteriosclerosis can be determined in a shorter time.

It should be noted that in the measurement device 1A and the measurement device 1B, the air bladder 13A serves not only for the purpose of avascularization but also for the purpose of calculation of blood pressure value. Then, the blood pressure value is calculated based on a change of the internal pressure of the air bladder 13A, and the pulse wave is measured based on a change of the internal pressure of the air bladder 13B. However, the air bladder 13A may be used only for avascularization, and the blood pressure value may be calculated based on a change of the internal pressure of the air bladder 13B.

### Third Embodiment

In some cases, it may be difficult to extract a feature point deriving especially from a reflection wave of the pulse wave (pulse wave 1) that is measured while the peripheral side of the measurement portion is avascularized to suppress the effect of the reflection wave. Accordingly, in the first embodiment and the second embodiment, the pulse wave (pulse wave 2) is measured in non-avascularized state in which the peripheral side is not avascularized, and the feature point is extracted from the pulse wave in the non-avascularized state. In this case, a pulse wave waveform is measured. The pulse wave waveform is a composite waveform made from an ejection wave emitted from the heart and a reflection wave emitted from a periphery such as a palm portion. However, a length from an upper arm, i.e., a measurement portion, to a palm is different for each subject. The length from the upper arm, i.e., the measurement portion, to the palm affects an arrangement between an ejection wave and a reflection wave, namely, the waveform of the measured pulse wave, i.e., the composite wave. Therefore, the accuracy of the obtained index is affected, and the determination of the degree of arteriosclerosis is also affected.

One method for suppressing this effect is as follows: the operation unit 3 and the like is used to input in advance a length between the upper arm, i.e., the measurement portion, and a position at which a large reflection occurs, i.e., the palm, and the measured pulse wave is corrected using the length. Another method is to fix the length between the measurement portion and the reflection position to a certain length.

Accordingly, in a measurement device 1C according to a third embodiment, the length between the measurement portion and the reflection position is fixed to a certain length, and another cuff to be attached to a periphery is arranged in addition to the air bladder for measurement process attached to the measurement portion in order to combine an ejection wave with a reflection wave emitted from the periphery located at the defined length from the measurement portion.

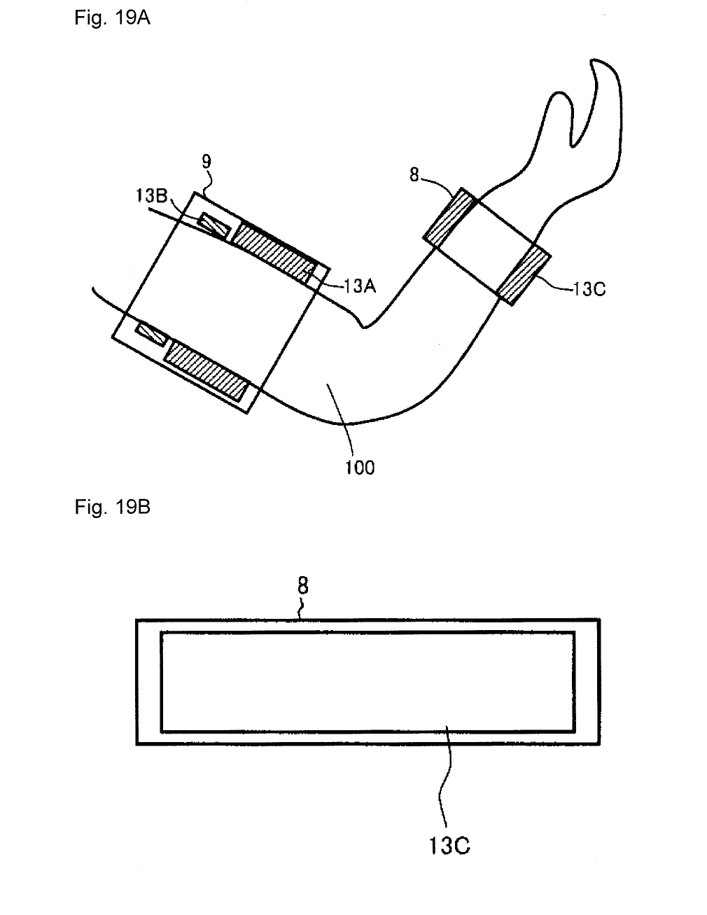

Referring to FIG. 19A, the measurement device 1C includes, for example, an arm band 8 to be wrapped around a wrist, i.e., a peripheral side with respect to the measurement portion. The arm band 8 includes an air bladder 13C as shown in FIG. 19B. As described above, the arm band 8 is attached to a wrist away by the predetermined length to the peripheral side from the arm band 9 including the air bladder 13A and the air bladder 13B. The attachment position may be determined by a person who carries out measurement. According to one or more embodiments of the present invention, a member for identifying the attachment position of the arm band 8, such as a belt having the predetermined length for connecting between the arm band 8 and the arm band 9, is included. The air bladder 13C inflates and pressurizes the wrist.

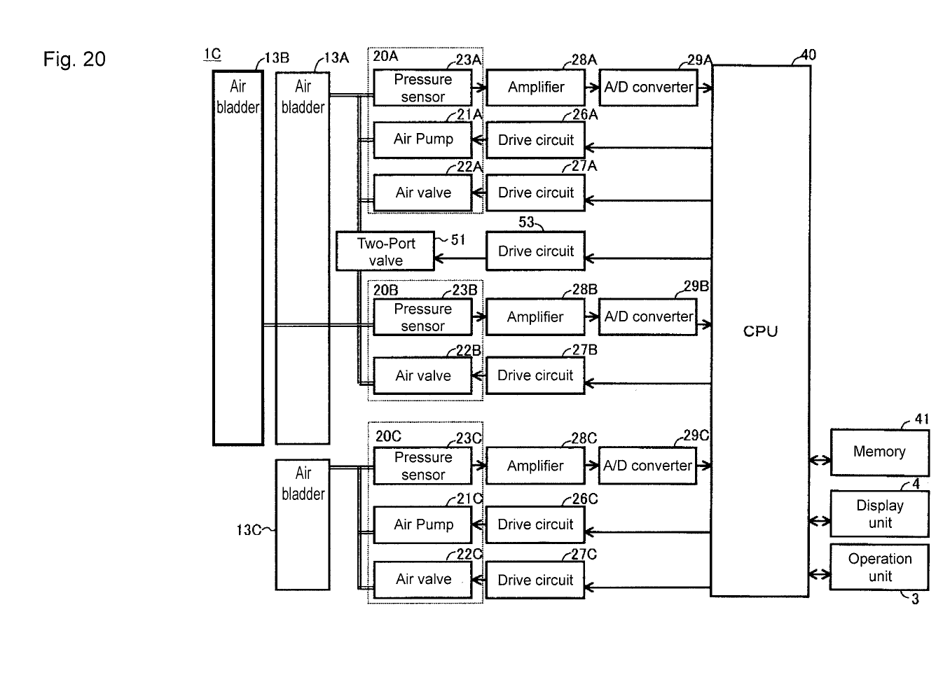

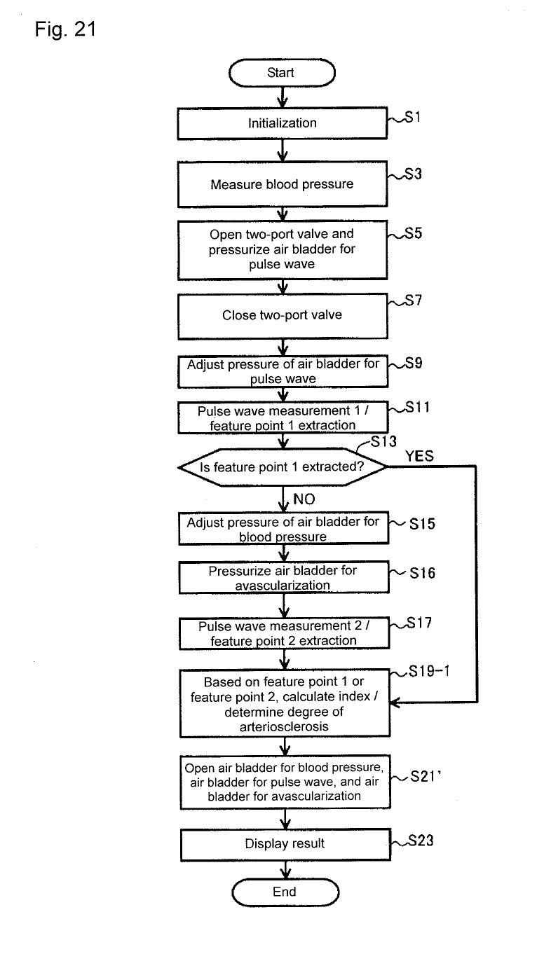

Referring to FIG. 20, the measurement device 1C includes an air system 20C connected to the air bladder 13C via an air tube in addition to the configuration of the measurement device 1A shown in FIG. 5.

The air system 20C includes an air pump 21C, an air valve 22C, and a pressure sensor 23C. The air pump 21C is driven by the drive circuit 26C receiving an instruction from the CPU 40, and blows compressed gas into the air bladder 13C. Thereby, the air bladder 13C is pressurized.

The open/close state of the air valve 22C is controlled by the drive circuit 27C receiving instructions from the CPU 40. The pressure in the air bladder 13C is controlled by controlling the open/close state of the air valves 22C.

The pressure sensor 23C detects the pressure in the air bladder 13C, and outputs a signal to an amplifier 28C according to the detected values thereof. The amplifier 28C amplifies the signal outputted from the pressure sensor 23C, and outputs the amplified signal to a converter 29C The converter 29C digitalizes analog signals outputted from the amplifier 28C, and outputs the digital signal to the CPU 40.

The CPU 40 controls the air systems 20A, 20B, 20C and the drive circuit 53 based on instructions inputted to the operation unit 3 on the base body 2 of the measurement device.

Further, according to one or more embodiments of the present invention, the measurement device 1C includes a device for inputting a length of an artery from the air bladder 13B to the air bladder 13C. The length of the artery from the air bladder 13B to the air bladder 13C may simply be a length of an arm from the air bladder 13B to the air bladder 13C, i.e., a length of the arm between the arm band 8 and the arm band 9. The device for inputting the length is not specifically limited. For example, the device may be a switch for inputting the length, included in the operation unit 3. When a person who carries out measurement inputs the length using the switch, the length is inputted. Alternatively, for example, the arm band 8 and the arm band 9 may be connected by a belt, and the device may be a mechanism arranged on the belt for detecting the length. By adjusting the length so as not to loosen the belt along the arm after the arm band 8 and the arm band 9 are attached, the length of the arm between the arm band 8 and the arm band 9 is inputted with the above mechanism.

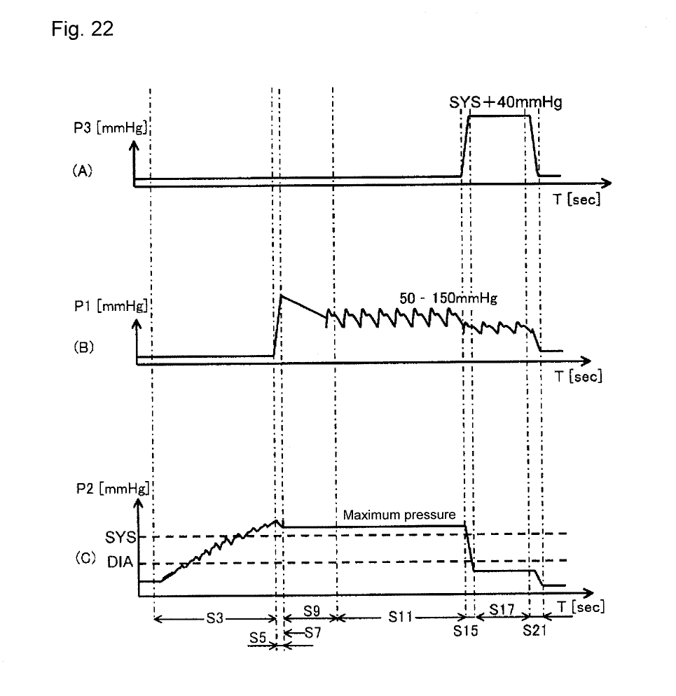

A first specific example of a measuring operation performed by the measurement device 1C will be described with reference to FIG. 21. The first specific example represents a measuring operation when calculation is performed according to the first arithmetic algorithm described in the first embodiment. The operation shown in FIG. 21 is started when a subject or the like presses down the measurement button on the operation unit 3 of the base body 2. This operation is achieved by the CPU 40. The CPU 40 reads a program stored in the memory 41 and controls each unit as shown in FIG. 20. In FIG. 22, a portion (A) represents a temporal change of a pressure P3 in the air bladder 13C, a portion (B) represents a temporal change of the pressure P1 in the air bladder 13B, and a portion (C) represents a temporal change of the pressure P2 in the air bladder 13A. In the portions (A), (B), (C) of FIG. 22, S3 to S21 attached to temporal axes correspond to respective operations of the measuring operation performed by the measurement device 1C.

Referring to FIG. 21, the measurement device 1C performs the same operation as steps S1 to S13 as the first specific example of the measuring operation performed by the measurement device 1A. As shown in (A) of FIG. 22, in the measurement device 1C, the pressure P3 in the air bladder 13C is maintained at an initial pressure during the process.

When the feature point 1 is not extracted from the pulse wave 1 during the avascularization in step S11 (NO in step S13), the CPU 40 reduces and adjusts the pressure P2 of the air bladder 13A so that the pressure P2 becomes lower than at least the systolic blood pressure, for example, about 55 mmHg in step S15, and outputs a control signal to the air system 20C, thereby increases the pressure P3 in the air bladder 13C so that the pressure P3 attains a predetermined pressure (step S16). In step S16, for example, the CPU 40 increases the pressure P3 to about the systolic blood pressure +40 mmHg, so that the pressure P3 becomes higher than at least the systolic blood pressure. At this time, the air bladder 13A does not avascularize an artery at the peripheral side close to the measurement portion, but the air bladder 13C avascularizes the artery at the position of the arm band 8 attached to the position away from the measurement portion by the predetermined length. Thereafter, the predetermined length at the peripheral side with respect to the measurement portion is not avascularized. At this state, the CPU 40 measures the pressure P1 in the air bladder 13B based on a pressure signal given by the pressure sensor 23B and thereby measures the pulse wave, thus extracting feature points in step S17. Thereafter, the same measuring operation as that of the measurement device 1A is performed.

Even when the second to fourth arithmetic algorithms described in the first embodiment are performed, the measuring operation of the measurement device 1C can be performed in the same manner.

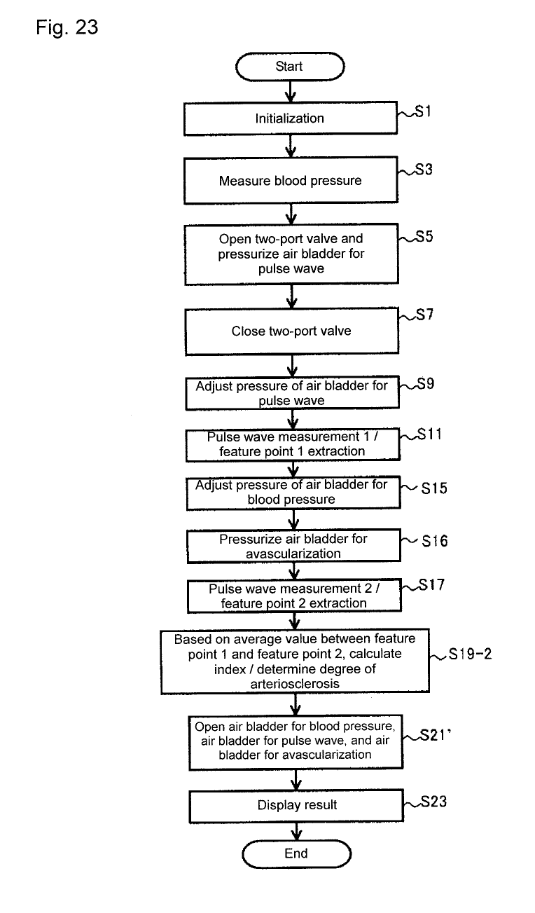

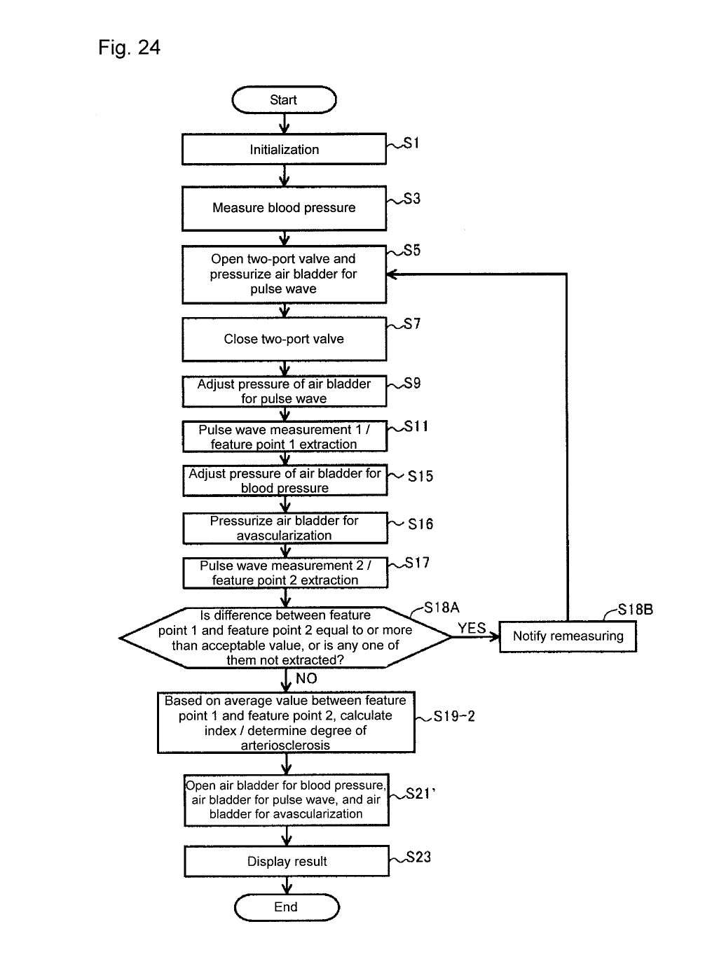

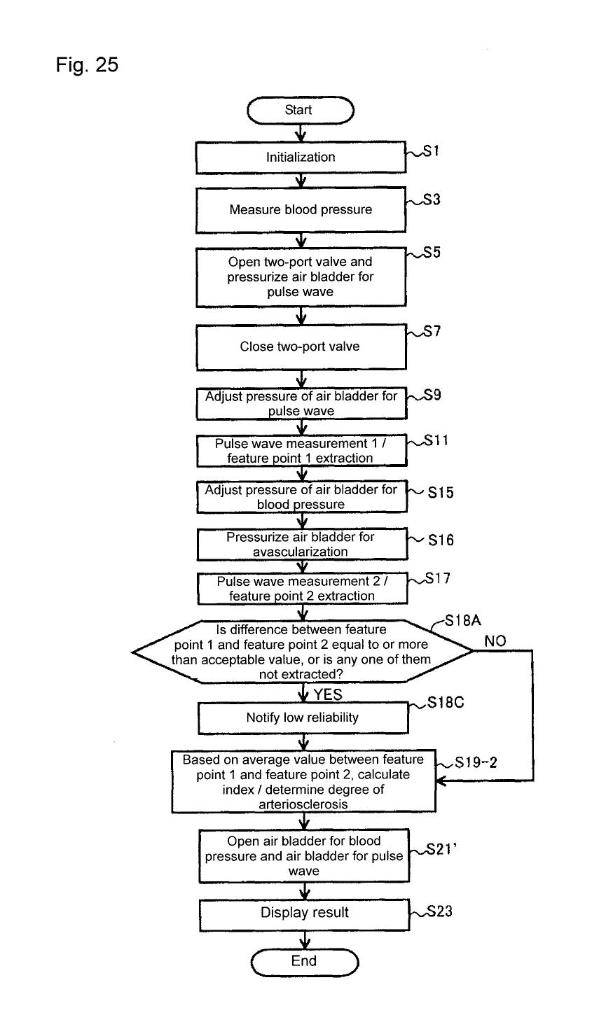

The second to fourth specific examples of the measuring operation performed by the measurement device 1C will be described with reference to FIG. 23 to FIG. 25. The measuring operations shown in these flowcharts are almost the same as the measuring operations according to the second to fourth specific examples performed by the measurement device 1A as shown in FIGS. 9 to 11, respectively. In any case, when the pulse wave 2 is measured in the non-avascularized state in step S17, the pressure P3 in the air bladder 13C is increased to a pressure higher than at least the systolic blood pressure in step S16, whereby the air bladder 13A does not avascularize the artery at the peripheral side close to the measurement portion but the air bladder 13C avascularizes the artery at the position of the arm band 8 attached to the position away from the measurement portion by the predetermined length.

The measurement device 1C achieves the measuring operations as shown in FIG. 21 and FIGS. 23 to 25. Accordingly, when the pulse wave (pulse wave 2) is measured in the non-avascularized state, the position at which the ejection wave is reflected can be adjusted. Therefore, the waveform of the pulse wave measured in the non-avascularized state is less affected by the length, which is different for each subject, from the measurement portion to the position at which the ejection wave is reflected. Therefore, the index can be more accurately calculated, and the index useful for determining the degree of arteriosclerosis can be obtained.

In the above example, an upper arm is the measurement portion, and the upper arm is attached with the arm band including the air bladder for avascularization of only the wrist corresponding to the position away from the upper arm by the predetermined length. Alternatively, when, for example, a plurality of reflection positions at the peripheral side are expected due to different measurement portions, a plurality of arm bands including respective air bladders for avascularization may be attached. In this manner, the index can be more accurately calculated.

In the above example, the measurement device 1C includes the air bladder 13C in addition to the configuration of the measurement device 1A. However, the measurement device 1C may include the air bladder 13C in addition to the configuration of the measurement device 1B. In this case, when the pressure P2 in the air bladder 13A becomes lower than the systolic blood pressure (NO in step S111) or when the pulse wave is measured during the pressure increasing process in step S104, the pressure P3 in the air bladder 13C is increased to a pressure higher than at least the systolic blood pressure, whereby the position away from the measurement portion by the predetermined length is avascularized.

It is to be understood that the embodiments disclosed herein are examples in all respects and are not restrictive. While the invention has been described with respect to a limited number of embodiments, those skilled in the art, having benefit of this disclosure, will appreciate that other embodiments can be devised which do not depart from the scope of the invention as disclosed herein. It is to be understood that the scope of the present invention is defined not by the above descriptions, but by the claims, and includes meanings equivalent to the claims and all the modifications and variations within the scope.

### DESCRIPTION OF SYMBOLS

1A, 1B, 1C Measurement device

2 Base body

3 Operation unit

4 Display unit

8, 9 Arm band

10 Air tube

13A, 13B, 13C Air bladder

20A, 20B, 20C Air system

21A, 21B, 21C Air pump

22A, 22B, 22C Air valve

23A, 23B, 23C Pressure sensor

26A, 26B, 26C, 27A, 27B, 27C, 53 Drive circuit

28A, 28B, 28C Amplifier

29A, 29B, 29C ND converter

31, 32 Switch

40 CPU

41 Memory

51 Two-port valve

100 Upper arm

1. A blood pressure information measurement device comprising:
a first fluid bag and a second fluid bag;
a first sensor and a second sensor for respectively measuring internal pressures of the first fluid bag and the second fluid bag;
a first adjusting unit for adjusting the internal pressure of the second fluid bag; and
a control unit for controlling calculation for calculating an index for determining a degree of arteriosclerosis and adjustment of the first adjusting unit,
wherein the control unit performs:
calculation for detecting a first pulse wave of a measurement portion based on a change of the internal pressure of the first fluid bag in a first state in which:
the first fluid bag is wrapped around the measurement portion;
the second fluid bag is wrapped at a peripheral side with respect to the first fluid bag; and
the second fluid bag presses the peripheral side with respect to the measurement portion around which the first fluid bag is wrapped with an internal pressure higher than a systolic blood pressure;
calculation for detecting a second pulse wave based on a change of the internal pressure of the first fluid bag in a second state in which:
the first fluid bag is wrapped around the measurement portion;
the second fluid bag is wrapped at a peripheral side with respect to the first fluid bag; and
the second fluid bag presses the peripheral side with respect to the measurement portion around which the first fluid bag is wrapped with an internal pressure lower than at least the systolic blood pressure; and
calculation for calculating the index using at least one of a first feature point extracted from the first pulse wave and a second feature point extracted from the second pulse wave.

2. A blood pressure information measurement device comprising:
a first fluid bag and a second fluid bag;
a first sensor and a second sensor for respectively measuring internal pressures of the first fluid bag and the second fluid bag;
a first adjusting unit for adjusting the internal pressure of the second fluid bag; and
a control unit for controlling calculation for calculating an index for determining a degree of arteriosclerosis and adjustment of the first adjusting unit,
wherein the control unit performs:
calculation for detecting a pulse wave of a measurement portion based on a change of the internal pressure of the first fluid bag in which:
the first fluid bag is wrapped around the measurement portion;
the second fluid bag is wrapped at a peripheral side with respect to the first fluid bag; and
the second fluid bag presses the peripheral side with respect to the measurement portion around which the first fluid bag is wrapped;
calculation for comparing a systolic blood pressure with the internal pressure of the second fluid bag when the pulse wave is detected, and determining whether the detected pulse wave is a first pulse wave detected in a first state in which the peripheral side of the measurement portion is pressed while the internal pressure of the second fluid bag is higher than the systolic blood pressure or a second pulse wave detected in a second state in which the peripheral side of the measurement portion is pressed while the internal pressure of the second fluid bag is lower than at least the systolic blood pressure; and
calculation for calculating the index using at least one of a first feature point extracted from the first pulse wave and a second feature point extracted from the second pulse wave.

3. The blood pressure information measurement device according to claim 1, wherein the control unit performs:
control of the first adjusting unit for attaining the first state by increasing the internal pressure of the second fluid bag to a pressure higher than at least the systolic blood pressure;
control of the first adjusting unit for reducing the internal pressure of the second fluid bag after increasing the internal pressure; and
calculation for extracting the second feature point from the second pulse wave detected in the second state in pressure reduction process and calculating the index using the second feature point in a case where the first feature point is not extracted from the first pulse wave detected in the first state.

4. A blood pressure information measurement device according to claim 1, wherein the index includes at least one of:
Tr (Traveling time to reflected wave) which is a time difference between an emerging time of a rise of an ejection wave and an emerging time of a rise of a reflection wave;
Tpp which is a time difference between an emerging time of a peak of the ejection wave and an emerging time of a peak of the reflection wave; and
AI (Augmentation Index) which is a ratio between an amplitude of the peak of the ejection wave and an amplitude of the peak of the reflection wave.

5. The blood pressure information measurement device according to claim 1, further comprising:
a third fluid bag; and
a second adjusting unit for adjusting an internal pressure of the third fluid bag,
wherein the control unit controls the second adjusting unit such that, in the second state, the internal pressure of the third fluid bag wrapped around a position away to the peripheral side from the measurement portion by a predetermined length attains a pressure higher than at least the systolic blood pressure, and the position away to the peripheral side from the measurement portion by the predetermined length is pressurized.

6. The blood pressure information measurement device according to claim 5, further comprising an input unit for inputting a length of a living body extending in the measurement portion from the first fluid bag wrapped around the measurement portion to the third fluid bag wrapped around the peripheral side with respect to the measurement portion.

7. The blood pressure information measurement device according to claim 1, further comprising an input unit for inputting a length from an upper arm serving as the measurement portion to a palm serving as a reflecting position of an ejection wave.

8. An index acquisition method for obtaining an index for determining a degree of arteriosclerosis from a pulse wave measured by a blood pressure information measurement device, wherein the blood pressure information measurement device includes:
a first fluid bag and a second fluid bag;
a first sensor and a second sensor for respectively measuring internal pressures of the first fluid bag and the second fluid bag; and
a first adjusting unit for adjusting the internal pressure of the second fluid bag, and
the index acquisition method includes the steps of:
controlling the internal pressure of the second fluid bag such that the internal pressure of the second fluid bag attains a pressure higher than a systolic blood pressure;
detecting a first pulse wave of a measurement portion based on a change of the internal pressure of the first fluid bag in a first state in which:
the first fluid bag is wrapped around the measurement portion;
the second fluid bag is wrapped at a peripheral side with respect to the first fluid bag; and
the second fluid bag presses the peripheral side with respect to the measurement portion around which the first fluid bag is wrapped with an internal pressure higher than the systolic blood pressure;
calculating the index from the first pulse wave;
performing control to reduce the internal pressure of the second fluid bag in a case where the index is not calculated from the first pulse wave;
detecting a second pulse wave of the measurement portion based on a change of the internal pressure of the first fluid bag in a state in which:
the first fluid bag is wrapped around the measurement portion;
the second fluid bag is wrapped at a peripheral side with respect to the first fluid bag; and
the second fluid bag presses the peripheral side with respect to the measurement portion with a pressure lower than at least the systolic blood pressure; and
calculating the index from the second pulse wave.

9. The blood pressure information measurement device according to claim 2, wherein the control unit performs:
control of the first adjusting unit for attaining the first state by increasing the internal pressure of the second fluid bag to a pressure higher than at least the systolic blood pressure;
control of the first adjusting unit for reducing the internal pressure of the second fluid bag after increasing the internal pressure; and
calculation for extracting the second feature point from the second pulse wave detected in the second state in pressure reduction process and calculating the index using the second feature point in a case where the first feature point is not extracted from the first pulse wave detected in the first state.

10. A blood pressure information measurement device according to claim 2, wherein the index includes at least one of:
Tr (Traveling time to reflected wave) which is a time difference between an emerging time of a rise of an ejection wave and an emerging time of a rise of a reflection wave;
Tpp which is a time difference between an emerging time of a peak of the ejection wave and an emerging time of a peak of the reflection wave; and
AI (Augmentation Index) which is a ratio between an amplitude of the peak of the ejection wave and an amplitude of the peak of the reflection wave.

11. The blood pressure information measurement device according to claim 2, further comprising:
a third fluid bag; and
a second adjusting unit for adjusting an internal pressure of the third fluid bag,
wherein the control unit controls the second adjusting unit such that, in the second state, the internal pressure of the third fluid bag wrapped around a position away to the peripheral side from the measurement portion by a predetermined length attains a pressure higher than at least the systolic blood pressure, and the position away to the peripheral side from the measurement portion by the predetermined length is pressurized.

12. The blood pressure information measurement device according to claim 11, further comprising an input unit for inputting a length of a living body extending in the measurement portion from the first fluid bag wrapped around the measurement portion to the third fluid bag wrapped around the peripheral side with respect to the measurement portion.

13. The blood pressure information measurement device according to claim 2, further comprising an input unit for inputting a length from an upper arm serving as the measurement portion to a palm serving as a reflecting position of an ejection wave.
